(19) United States
(12) Patent Application Publication
(10) Pub. No.: US 2022/0071999 A1
(43) Pub. Date: Mar. 10, 2022

(54) DESIGN AND DISCOVERY OF BD OXIDASE INHIBITORS FOR THE TREATMENT OF MYCOBACTERIAL DISEASES

(71) Applicants: UNIVERSITY OF NOTRE DAME DU LAC, SOUTH BEND, IN (US); MONTANA STATE UNIVERSITY, BOZEMAN, MT (US); NANYANG TECHNOLOGICAL UNIVERSITY, Singapore (SG)

(72) Inventors: Marvin J. MILLER, South Bend, IN (US); Garrett C. MORASKI, Bozeman, MT (US); Kevin PETHE, Singapore (SG)

(73) Assignees: UNIVERSITY OF NOTRE DAME DU LAC, SOUTH BEND, IN (US); MONTANA STATE UNIVERSITY, BOZEMAN, MT (US); NANYANG TECHNOLOGICAL UNIVERSITY, SINGAPORE, SG (SG)

(21) Appl. No.: 17/416,016

(22) PCT Filed: Dec. 19, 2019
(86) PCT No.: PCT/IB2019/061142

§ 371 Date: Jun. 18, 2021

Related U.S. Application Data
(60) Provisional application No. 62/783,984, filed on Dec. 21, 2018.

(51) Int. Cl. A61K 31/517; A61K 45/06; C07D 239/94; A61P 31/04

(52) U.S. Cl. CPC A61K 31/517; A61P 31/04; C07D 239/94; A61K 45/06

(57) ABSTRACT

Described are compounds and compositions that inhibit or target Cyt-bd, and methods of making, using, and assaying same. Also disclosed are compositions, methods and kits including the compounds and compositions that inhibit or target Cyt-bd, and one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent.

### RELATED APPLICATIONS

This application claims the benefit of U.S. Application No. 62/783,984, filed Dec. 21, 2018, the entire contents of which are hereby incorporated by reference.

### GOVERNMENT SUPPORT

This invention was made with government support under Grant No. R37 AI054193 awarded by the National Institutes of Health. The United States Government has certain rights in the invention.

### BACKGROUND

Mycobacterium Tuberculosis (Mtb) caused 1.8 million deaths and 10.4 million new infections in 2015. Throughout history, TB has claimed the lives of over one billion people and currently infects one third of the world's population. The increase in cases of TB/HIV co-infection and the spread of multiple-drug resistant TB (MDR-TB, strains that are resistant to first line drugs isoniazid and rifampin) and extensively drug resistant TB (XDR-TB, strains that are resistant to isoniazid and rifampin, as well as any fluoroquinolone and at least one of three injectable second-line drugs, such as amikacin, kanamycin, or capreomycin) are making matters worse. The proportion of multi-drug resistant (MDR)-TB among newly diagnosed cases is a staggering 5.6%, mostly due to non-compliance with the standard, six-month drug regimen. There is therefore a pressing clinical need for new drugs with short treatment courses. Rational combinations of synergistic agents would thwart the emergence of MDR- and extensively-drug resistant (XDR)-TB. The recent approval of bedaquiline (BDQ, Sirturo®) and Delamanid (Deltyba®) represents a critical milestone in anti-TB drug discovery. Unfortunately, initial gains by BDQ were quickly overshadowed by the emergence of resistance less than 3 years after its introduction into clinical practice. This is probably linked to the absence of potent companion drugs. Indeed, BDQ is currently given in combination with weaker, second and third line drugs, a regimen that strongly selects for BDQ resistance. Combinations of synergistic drugs with shortened treatment duration are required to lower the incidence of resistance.

BDQ is a potent inhibitor of the mycobacterial F₁F₀-ATP synthase. Its discovery and clinical development has validated the electron transport chain of Mtb as a viable drug target. Several new drug candidates (e.g. Q203) that inhibit the cytochrome (Cyt) bc₁:aa₃ complex of Mtb are in the pipeline. However, all bc₁ inhibitors are bacteriostatic in Mtb. The scientific premise of this patent is that the lack of cidal activity by this class of drugs is due to the presence of a second terminal oxidase in Mtb, the so-called cytochrome bd oxidase (Cyt-bd). In addition to its role as a terminal oxygen reductase, Cyt-bd is also required in cellular redox buffering that occurs in response to redox stressors (e.g. oxidative and nitrosative stress, antibiotics).

Combination therapy is now the standard in all TB treatment regimens. Currently, these are an amalgam of monotherapies whose combinatorial effects were only examined at the clinical testing stage. By screening for synthetic lethality at early stages of drug discovery, the chances of finding more efficacious synergistic drug cocktails will be significantly increased. Developing effective combination therapies has the potential to decrease both deleterious side effects and the incidence of antibiotic resistance in TB. The present inventors and others have previously suggested that interference with OxPhos at multiple levels is a promising anti-TB strategy.

### BRIEF DESCRIPTION OF THE FIGURES

The Figures are attached and made part of this document.

FIGS. 1a-b present data for exemplary Cyt-bd inhibitor screening assay in mycobacteria.

FIGS. 2a-b present data for exemplary Cyt-bd inhibitor screening assay in mycobacteria.

FIGS. 3a-b present data for exemplary Cyt-bd inhibitor screening assay in mycobacteria.

FIG. 4 presents data for exemplary Cyt-bd inhibitor screening assay in mycobacteria.

FIG. 5 presents exemplary 6,6-compounds and two comparative compounds (ND-011987 and ND-012030 are comparative).

FIG. 6 presents MICs of exemplary 6,6-compounds and comparative compounds.

FIG. 7 presents exemplary 5,6-compounds and a comparative compound (ND-011986 is comparative).

FIG. 8 presents MICs of exemplary 5,6-compounds and comparative compounds.

Each and every compound set out in the Figures is made a part of the specification description, the same as if set forth at length.

### BRIEF DESCRIPTION

One embodiment provides a compound described herein that inhibits or targets Cyt-bd.

Another embodiment provides a pharmaceutical composition comprising the above compound and a pharmaceutically acceptable carrier.

Another embodiment provides a compound having any one of the formulas (A)-(E), (A′), (A″), and (B′) described herein, or pharmaceutically acceptable salt thereof.

Another embodiment provides a pharmaceutical composition comprising the above compound and a pharmaceutically acceptable carrier.

Another embodiment provides a kit, comprising:
the above compound or composition; and
one or more Cyt-bc1:aa3 inhibitor or F₁F₀-ATP synthase inhibitor, or
one or more pharmaceutical composition comprising one or more Cyt-bc1: aa3 inhibitor or F₁F₀-ATP synthase inhibitor and a pharmaceutically acceptable carrier.

Another embodiment provides a method, comprising administering the above compound or composition or kit, to a subject suffering from mycobacterial disease or infection.

Another embodiment provides a method, comprising co-administering, to a subject suffering from mycobacterial disease or infection:

the above compound or composition; and

one or more Cyt-bc1:aa3 inhibitor or F₁F₀-ATP synthase inhibitor, or one or more pharmaceutical composition comprising one or more Cyt-bc1:aa3 inhibitor or F₁F₀-ATP synthase inhibitor and a pharmaceutically acceptable carrier.

Another embodiment provides a method, comprising co-administering, to a subject suffering from mycobacterial disease or infection:

the above compound or composition; and

one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, or

one or more pharmaceutical composition comprising one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, and a pharmaceutically acceptable carrier.

Another embodiment provides a kit, comprising:

the above compound or composition; and

one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, or

one or more pharmaceutical composition comprising one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, and a pharmaceutically acceptable carrier.

### DETAILED DESCRIPTION

The present inventors have found that compounds that inhibit Cyt-bd are particularly useful to unleash the full potential of drugs that inhibit the Cyt-bc1:aa3 branch of oxidative phosphorylation (sometimes called Cyt-bc1:aa3 inhibitors herein) and/or drugs that inhibit the mycobacterial F₁F₀-ATP synthase (sometimes called F₁F₀-ATP synthase inhibitors herein). The present inventors have developed Cyt-bd inhibitor compounds and found that when administered in concert with Cyt-bc1:aa3 inhibitors and/or F₁F₀-ATP synthase inhibitors, an effective treatment of MDR-TB and XDR-TB is obtained. The present inventors have developed an assay/screening protocol to determine which compounds inhibit Cyt-bd. The present inventors have found that a rationally-designed drug combination simultaneously targeting the Cyt-bc1:aa3, the Cyt-bd, and/or F₁F₀-ATP synthase may be the cornerstone of a sterilizing drug combination for the treatment of MDR- and XDR-TB.

The difficulty in identifying drugs that target Cyt-bd lies in the non-essentiality of the target. Indeed, the genes encoding for Cyt-bd can be deleted without obvious phenotypes on growth, ATP homeostasis, or respiration. However, respiration through the Cyt-bd branch becomes essential upon chemical inhibition of the Cyt-bc1:aa3 branch. The present inventors have found for the first time a cell-based drug screen for identification of inhibitors of Cyt-bd that synergize with known Cyt-bc1:aa3 inhibitors (like Q203). This screening protocol described herein yielded multiple inhibitors of classes of compounds that target and inhibit Cyt-bd.

Many Cyt-bc1:aa3 inhibitors are known. See, for example, Abrahams, K. A. et al. Identification of novel imidazo[1,2-a]pyridine inhibitors targeting M. tuberculosis QcrB. PLoS One 7, e52951 (2012); Kang, S. et al. Lead optimization of a novel series of imidazo[1,2-a]pyridine amides leading to a clinical candidate (Q203) as a multi- and extensively-drug-resistant anti-tuberculosis agent. J Med Chem 57, 5293-305 (2014); Moraski, G. C. et al. Advent of Imidazo[1,2-a]pyridine-3-carboxamides with Potent Multi- and Extended Drug Resistant Antituberculosis Activity. ACS Med Chem Lett 2, 466-470 (2011); and Pethe, K. et al. Discovery of Q203, a potent clinical candidate for the treatment of tuberculosis. Nat Med 19, 1157-60 (2013). See also U.S. Pat. No. 9,309,238, issued Apr. 12, 2016, U.S. Patent Application Publication 2018/0265506, published Sep. 20, 2018, U.S. Pat. No. 9,605,002, issued Mar. 28, 2017, and U.S. Application 62/641,668, entitled, “Deuterated Imidazopyridines”, filed Mar. 12, 2018.

Many F₁F₀-ATP synthase inhibitors are known. See, for example, reference 2 herein and and Riccardi, N. et. al. Bedaquiline: A New Hope for Shorter and Better Anti-Tuberculosis Regimens. Recent Pat Antiinfect Drug Discov. 13(1), 3-11 (2018).

Mycobacteria energetics, e.g., ATP synthase, cyt-bc1:aa3 and cyt-bd oxidase are discussed in reference 10 herein and and Kumar, A. et. al. Bioenergetics of Mycobacterium: An Emerging Landscape for Drug Discovery. Pathogens 7(1), 24 (2018); doi:10.3390/pathogens7010024.

Herein the present inventors show that (1) combined inhibition of Cyt-bd and Cyt-bc₁:aa₃ will abrogate terminal oxidation in Mtb even under hypoxic conditions and (2) inhibition of Cyt-bd will enhance efficacy of other anti-TB drugs to eradicate infection. The data presented herein show that Cyt-bd allows maintenance of respiration in the absence of Cyt-bc₁:aa₃, while simultaneous inhibition of both oxidases leads to rapid killing and clearance of Mtb during acute infection in mouse lungs. It has now been found that Cyt-bd is employed by Mtb to overcome redox stress under hypoxia and antibiotic challenge, thereby contributing to its extraordinary ability for long-term persistence and drug tolerance in the host.

The invention provides a series of compounds that target and inhibit Cyt-bd: quinazoline, quinoline, thieno[3,2-d]pyrimidin-4-amine, furo[3,2-d]pyrimidin-4-amine, 5H-pyrrolo[3,2-d]pyrimidin-4-amine, and 7H-purin-6-amine compounds, syntheses thereof, compositions thereof, and methods of using such compounds and compositions.

As used herein, the quinazoline quinoline compounds are sometimes referred to herein as “6,6-compounds”. As used herein, the thieno[3,2-d]pyrimidin-4-amine, furo[3,2-d]pyrimidin-4-amine, 5H-pyrrolo[3,2-d]pyrimidin-4-amine, and 7H-purin-6-amine compounds are sometimes referred to herein as “5,6-compounds”.

Bacterial Strains, Diseases, and Subject Patients

Various embodiments provide compounds, compositions and methods of killing and/or inhibiting the growth of bacteria including Tuberculous mycobacteria species (such as Mycobacterium tuberculosis) and Non-Tuberculous Mycobacteria species including, but not limited to: Mycobacterium avium, Mycobacterium abscessus, Mycobacterium paratuberculosis, Mycobacterium kansasii, Mycobacterium ulcerans, Mycobacterium marinum, Mycobacterium intracellularae and other species thereof described as causing human and/or animal disease.

The subject may be human or animal.

BD Oxidase Assay

Targeting respiration and ATP synthesis has received strong interest as a new strategy for combatting Mycobacterium tuberculosis and other health relevant mycobacteria. Mycobacteria employ an aerobic respiratory chain terminating with two branches. One of the branches is the cytochrome bc1 aa3-type cytochrome c oxidase supercomplex, while the other branch terminates with a cytochrome bd-type quinol oxidase (the cytochrome bd oxidase). The assay evaluates the potency of compounds against the cytochrome bd-type quinol oxidase. The assay principle relies on the essentiality of the cytochrome bd oxidase to maintain ATP homeostasis when the cytochrome bc1:aa3 branch is inhibited by with a specific small-molecule inhibitor (Q203). Upon inhibition of the cytochrome bc1:aa3 branch with 100 nM of Q203, putative cytochrome bd oxidase are tested in a dose-response for their capacity to inhibit ATP synthesis after 12 to 16 hours of incubation. The IC50 values are determined for the test compound in the presence of Q203 (“+Q203”) or in the absence of Q203 (“-Q203”). Validated small-molecule inhibitors of the cytochrome bd oxidase inhibit ATP homeostasis in the presence of a cytochrome bc1:aa3 inhibitor (for example Q203, ND-11598, ND-11176, TB47, others as shown herein), other cytochrome bc1:aa3 inhibitor, other imidazopyridine carboxamides, or other classes of cytochrome c oxidase inhibitors.

BD Oxidase Inhibitor Compounds of Formula (A):

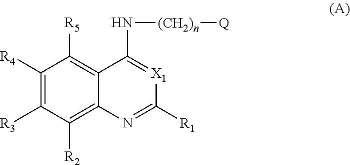

wherein

X₁ is CH, CR₆, or N;

R₁ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₂ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₃ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₄ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₅ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₆ is H, D, halogen, alkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

n=0, 1, 2, 3 or 4;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of alkyl, alkenyl, aryl, bi-aryl, bi-aryl ether, heteroaryl, heterocycle, cyclic amine, alkynyl, propargyl, triazole, polyethylene glycol, or any of the Q's hereinbelow, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched.

If desired, in the compound of Formula (A) above and in formulas (A′), (B)-(E) below, each of R₁, R₂, R₃, R₄, R₅, and R₆ may be H. If desired, each of R₁, R₂, R₃, R₄, R₅, and R₆ may be D. If desired, the R₁, R₂, R₃, R₄, R₅, and R₆ may a combination of H and D. It is also contemplated that not all of R₁, R₂, R₃, R₄, R₅, and R₆ are simultaneously H or D, and in such a case, one or more than one of R₁, R₂, R₃, R₄, R₅, and R₆ may be suitably selected from the substituents given, e.g., halogen, alkyl, cycloalkyl, F, alkoxy, and so on. For example, say, when R₁ is H, D, then R₂=halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, or COOR′. As a further example, which is not intended to be limiting, if R₄ is F or CH₃, then R₂, R₃, and R₅ are H or D, or a combination thereof, and so on. And though not always, it is usually the case that only one or two of R₁, R₂, R₃, R₄, R₅, and R₆ are not H or D.

For example, the compound of formula (A) may have one of the following formulas:

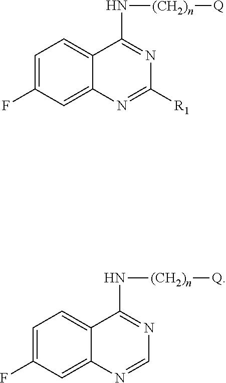

Other examples of the compound having formula (A) include:

BD Oxidase Inhibitor Compounds of Formula (A′):

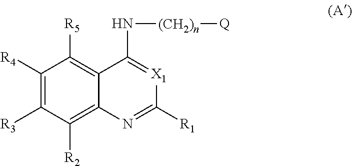

wherein

X₁ is CH, CR₆, or N;

R₁ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile, carboxy;

R₂ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₃ is H, D, halogen, alkyl, alkenyl, alkynyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₄ is H, D, halogen, alkyl, alkenyl, alkynyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₅ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₆ is H, D, halogen, alkyl, alkenyl, alkynyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

n=0, 1, 2, 3 or 4;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of alkyl, alkenyl, aryl, bi-aryl, bi-aryl ether, heteroaryl, heterocycle, cyclic amine, alkynyl, propargyl, triazole, polyethylene glycol, or any of the Q's hereinbelow, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched;

provided that when R¹ is substituted or unsubstituted alkyl or substituted or unsubstituted alkenyl, Q is not substituted phenyl or unsubstituted phenyl.

Other examples of the compound having formula (A) include:

BD Oxidase Inhibitor Compounds of Formula (A″):

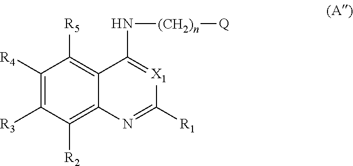

wherein

X₁ is CH, CR₆, or N;

R₁ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₂ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₃ is halogen;

R₄ is H, D, halogen, alkyl, alkenyl, alkynyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₅ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₆ is H, D, halogen, alkyl, alkenyl, alkynyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

n=0;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is bi-aryl ether, wherein said bi-aryl ether is independently substituted or unsubstituted;

provided that when R¹ is substituted or unsubstituted alkyl or substituted or unsubstituted alkenyl, Q is not substituted phenyl or unsubstituted phenyl.

If desired, in the compound of Formula (A″) above, each of R₁, R₂, R₄, R₅, and R₆ may be H. If desired, each of R₁, R₂, R₄, R₅, and R₆ may be D. If desired, the R₁, R₂, R₄, R₅, and R₆ may a combination of H and D. It is also contemplated that not all of R₁, R₂, R₄, R₅, and R₆ are simultaneously H or D, and in such a case, one or more than one of R₁, R₂, R₄, R₅, and R₆ may be suitably selected from the substituents given, e.g., halogen, alkyl, cycloalkyl, F, alkoxy, and so on.

BD Oxidase Inhibitor Compounds of Formula (B):

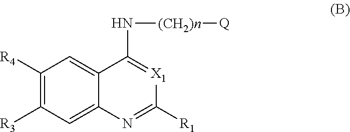

wherein

X₁ is CH, CR₆, or N;

R₁ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₃ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₄ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₆ is H, D, halogen, alkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

n=0, 1, 2, 3 or 4;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of alkyl, alkenyl, aryl, bi-aryl, bi-aryl ether, heteroaryl, heterocycle, cyclic amine, alkynyl, propargyl, triazole, polyethylene glycol, or any of the Q's hereinbelow, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched.

Other examples of the compound having formula (B) include:

BD Oxidase Inhibitor Compounds of Formula (B′):

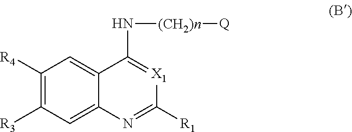

wherein

X₁ is N;

R₁ is H, D, halogen, alkyl, alkenyl, alkynyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₃ is halogen;

R₄ is H, D, halogen, alkyl, alkenyl, alkynyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

R₆ is H, D, halogen, alkyl, alkenyl, alkynyl, CH₃, alkoxy, CF₃, OCF₃, SF₃, SF₅, PO(CH₃)₂, COR′, CONH₂, CONHR′, CONR′R″, NH₂, NHR′, NR′R″, NHCOR′, NR′COR″, COOR′, nitrile;

n=0;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of aryl, bi-aryl, bi-aryl ether, heteroaryl, heterocycle, cyclic amine, or any of the Q's hereinbelow, alone or in any combination, wherein said any of the aryl, bi-aryl, bi-aryl ether, heteroaryl, heterocycle, cyclic amine or combination thereof is independently substituted or unsubstituted,
provided that when R¹ is substituted or unsubstituted alkyl or substituted or unsubstituted alkenyl, Q is not substituted phenyl or unsubstituted phenyl.

BD Oxidase Inhibitor Compounds of Formula (C):

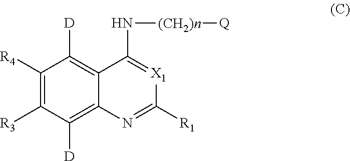

wherein

X₁ is CH, CR₆, or N;

R₁ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₃ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₄ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₆ is H, D, halogen, alkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

n=0, 1, 2, 3 or 4;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of alkyl, alkenyl, aryl, bi-aryl, bi-aryl ether, heteroaryl, heterocycle, cyclic amine, alkynyl, propargyl, triazole, polyethylene glycol, or any of the Q's hereinbelow, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched.

BD Oxidase Inhibitor Compounds of Formula (D):

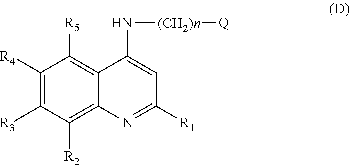

wherein

R₁ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₂ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₃ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₄ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

R₅ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₆ is H, D, halogen, alkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

n=0, 1, 2, 3 or 4;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of alkyl, alkenyl, aryl, bi-aryl, bi-aryl ether, heteroaryl, heterocycle, cyclic amine, alkynyl, propargyl, triazole, polyethylene glycol, or any of the Q's hereinbelow, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched.

BD Oxidase Inhibitor Compounds of Formula (E):

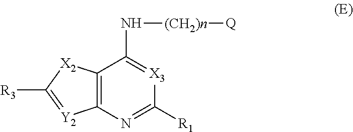

wherein

X₂ is S, O, CH₂, CHR₁₂, NH, NR₁₃; wherein R₁₂ is independently alkyl, halogen, alkoxy, CF₃, OCF₃; and wherein Rn is independently alkyl;

Y₂ is CH, CR₁₂, N; wherein Ru is independently alkyl, halogen, alkoxy, CF₃, OCF₃; R₁ is H, D, halogen, alkyl, cycloalkyl, CH₃, alkoxy, CF₃, OCF₃, SF₃, SF₅, PO(CH₃)₂, COR′, CONH₂, CONHR′, CONR′R″, NH₂, NHR′, NR′R″, COOR′;

R₃ is H, D, halogen, alkyl, cycloalkyl, F, alkoxy, CF₃, OCF₃, SF₃, SF₅, PO(CH₃)₂, COR′, CONH₂, CONHR′, CONR′R″, NH₂, NHR′, NR′R″, COOR′;

n=0, 1, 2, 3 or 4;

wherein R′ is independently C₁₋₆ alkyl, C₃₋₈ cycloalkyl, C₆₋₁₂ aryl, or C₅₋₆ heteroaryl;

wherein R″ is independently C₁₋₆ alkyl, C₃₋₈ cycloalkyl, C₆₋₁₂ aryl, or C₅₋₆ heteroaryl;

wherein any of the alkyl, CH₃, cycloalkyl, alkoxy, in any R group is each independently substituted or unsubstituted, branched or unbranched, or any combination thereof;

and

Q is any of alkyl, alkenyl, aryl, bi-aryl, bi-aryl ether, heteroaryl, heterocycle, cyclic amine, alkynyl, propargyl, triazole, polyethylene glycol, or any of the Q's hereinbelow, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched.

For example, compound (E) may have one of the following formulas:

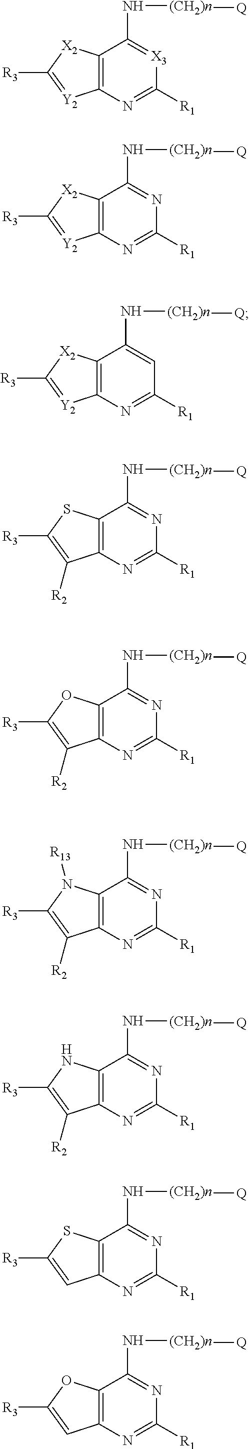

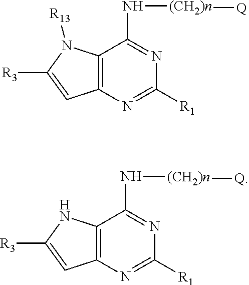

Other examples of the compound having formula (E) include:

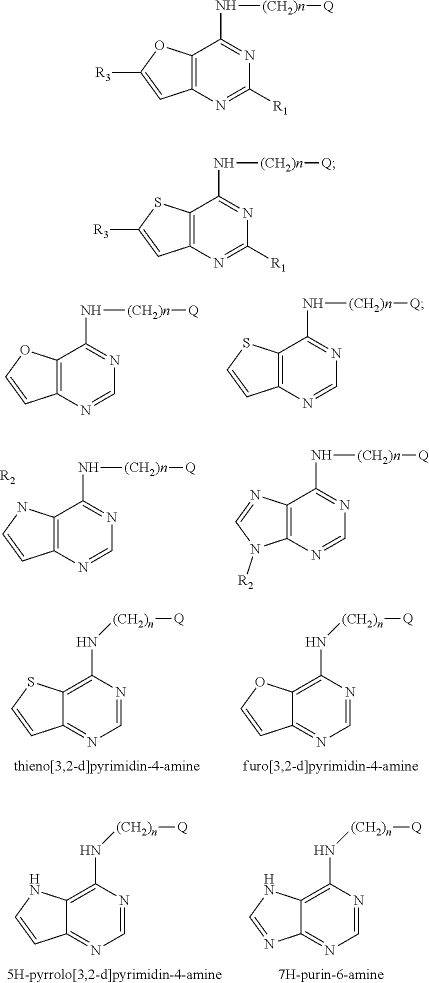

Exemplary “Q” Portions of BD Oxidase Inhibitors

In any embodiment herein, in any one of the compounds of Formulas (A)-(E), (A′), (A″) and (B′), Q may be any of alkyl, alkenyl, aryl, bi-aryl, bi-aryl ether, heteroaryl, heterocycle, cyclic amine, alkynyl, propargyl, triazole, polyethylene glycol, alone or in any combination, wherein said any of the alkyl, alkenyl, aryl, bi-aryl, bi-aryl ether, heteroaryl, heterocycle, cyclic amine or combination thereof is independently substituted or unsubstituted, and wherein said any of the alkyl, alkenyl, or combination thereof is independently branched or unbranched.

In embodiments, Q may be independently unsubstituted or substituted with C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, aryl, heteroaryl, cyclic amine, cycloalkyl, heterocycle, or combination thereof.

In embodiments, Q may be independently unsubstituted or substituted with alkyl, alkoxy, halogen, hydroxyl, CF₃, OCF₃, SF₅, SF₃, sulfate, nitrate, carboxylate, carboxylic acid, aryl, heteroaryl or any combination thereof.

In any embodiment herein, Q may be any of alkyl, alkenyl, terpene, geranyl, geranylgeranyl, farnesyl;

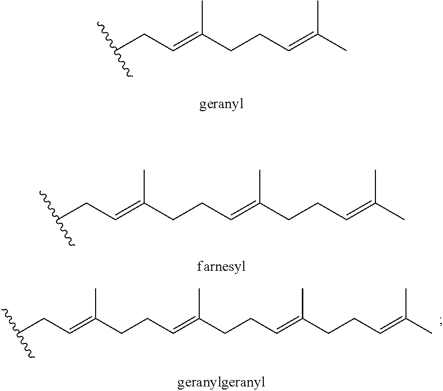 [SMILES: *C/C=C(\C)CCC=C(C)C ; *C/C=C(\C)CC/C=C(\C)CCC=C(C)C ; *C/C=C(\C)CC/C=C(\C)CC/C=C(\C)CCC=C(C)C]

wherein any of said alkyl, alkenyl, terpene, geranyl, geranylgeranyl, farnesyl are substituted or unsubstituted; and wherein said alkyl or alkenyl may be branched or unbranched.

In embodiments, Q may be independently unsubstituted or substituted with C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, aryl, heteroaryl, cyclic amine, cycloalkyl, heterocycle, or combination thereof.

In embodiments, Q may be independently unsubstituted or substituted with alkyl, alkoxy, halogen, hydroxyl, CF₃, OCF₃, SF₅, SF₃, sulfate, nitrate, carboxylate, carboxylic acid, carboxamide, aryl, heteroaryl or any combination thereof.

In any embodiment herein, Q may have the following formula:

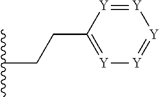

wherein each Y is independently CH, CR₇, or N, wherein at most two Ys are N; each R₇ is independently H, D, alkyl, t-butyl, alkenyl, isopropoxy, cycloalkyl, cyclic amine, CF₃, OCF₃, SF₅, SF₃, halo, methylsulfone, alkoxy, amine, nitrile, aryl, heteroaryl, heterocycle, furan, thiophene, oxazole, isoxazole, imidazole, pyrrole, pyrrolidine, tetrahydrofuran, pyridine, piperidine, pyrimidine, pyrazine, azepine, 1,4-diazepine, 4H-pyran, tetrahydropyran, indole, quinoline, isoquinoline, chroman, purine, pteridine, benzimidazole, benzothiazole, benzoxazole, benzofuran, benzothiazole, or 1H-indazole; in which each R₇ may be independently branched or unbranched, substituted or unsubstituted, or combination thereof.

In embodiments, each R₇ may suitably and independently be H, D, alkyl, t-butyl, alkenyl, isopropoxy, cycloalkyl, cyclic amine, CF₃, OCF₃, SF₅, SF₃, halo, methylsulfone, alkoxy, amine, nitrile, aryl, or heteroaryl; wherein any of the alkyl, t-butyl, alkoxy, isopropoxy is each independently substituted or unsubstituted, branched or unbranched, or any combination thereof.

In embodiments, each R₇ may suitably and independently be H, D, alkyl, t-butyl, isopropoxy, CF₃, OCF₃, SF₅, SF₃, halo, methylsulfone, alkoxy, amine, nitrile; wherein any of the alkyl, t-butyl, alkoxy, isopropoxy is each independently substituted or unsubstituted, branched or unbranched, or any combination thereof.

In embodiments, each R₇ may be independently H, D, alkyl, t-butyl, isopropoxy, CF₃, OCF₃, SF₅, SF₃, halo, methylsulfone, alkoxy, amine, or nitrile; and wherein any of the alkyl, t-butyl, alkoxy, isopropoxy in any R₇ is each independently substituted or unsubstituted, branched or unbranched, or any combination thereof.

In embodiments, each R₇ may be independently unsubstituted or substituted with C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, aryl, heteroaryl, cyclic amine, cycloalkyl, heterocycle, or combination thereof.

In embodiments, each R₇ may be independently unsubstituted or substituted with alkyl, alkoxy, halogen, hydroxyl, CF₃, OCF₃, SF₅, SF₃, sulfate, nitrate, carboxylate, carboxylic acid, aryl, heteroaryl or any combination thereof.

In any embodiment herein, Q may have the following formula:

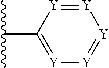

wherein each Y is independently CH, CR₇, or N, wherein at most two Ys are N; each R₇ is independently H, D, alkyl, t-butyl, alkenyl, isopropoxy, cycloalkyl, cyclic amine, CF₃, OCF₃, SF₅, SF₃, halo, methylsulfone, alkoxy, amine, nitrile, aryl, heteroaryl, heterocycle, furan, thiophene, oxazole, isoxazole, imidazole, pyrrole, pyrrolidine, tetrahydrofuran, pyridine, piperidine, pyrimidine, pyrazine, azepine, 1,4-diazepine, 4H-pyran, tetrahydropyran, indole, quinoline, isoquinoline, chroman, purine, pteridine, benzimidazole, benzothiazole, benzoxazole, benzofuran, benzothiazole, or 1H-indazole; in which each R₇ may be independently branched or unbranched, substituted or unsubstituted, or combination thereof.

In embodiments, each R₇ may suitably and independently be H, D, alkyl, t-butyl, alkenyl, isopropoxy, cycloalkyl, cyclic amine, CF₃, OCF₃, SF₅, SF₃, halo, methylsulfone, alkoxy, amine, nitrile, aryl, or heteroaryl; wherein any of the alkyl, t-butyl, alkoxy, isopropoxy is each independently substituted or unsubstituted, branched or unbranched, or any combination thereof.

In embodiments, each R₇ may suitably and independently be H, D, alkyl, t-butyl, isopropoxy, CF₃, OCF₃, SF₅, SF₃, halo, methylsulfone, alkoxy, amine, nitrile; wherein any of the alkyl, t-butyl, alkoxy, isopropoxy is each independently substituted or unsubstituted, branched or unbranched, or any combination thereof.

In embodiments, each R₇ may be independently H, D, alkyl, t-butyl, isopropoxy, CF₃, OCF₃, SF₅, SF₃, halo, methylsulfone, alkoxy, amine, or nitrile; and wherein any of the alkyl, t-butyl, alkoxy, isopropoxy in any R₇ is each independently substituted or unsubstituted, branched or unbranched, or any combination thereof.

In embodiments, each R₇ may be independently unsubstituted or substituted with C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, aryl, heteroaryl, cyclic amine, cycloalkyl, heterocycle, or combination thereof.

In embodiments, each R₇ may be independently unsubstituted or substituted with alkyl, alkoxy, halogen, hydroxyl, CF₃, OCF₃, SF₅, SF₃, sulfate, nitrate, carboxylate, carboxylic acid, aryl, heteroaryl or any combination thereof.

In any embodiment herein, Q may have the following formula:

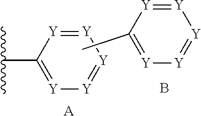

wherein each Y in the A ring is independently CH, CR₇, N, or C-B ring; wherein at most two Ys in the A ring are N; wherein at most two Ys in the A ring are independently C-B ring; each R₇ is independently H, D, alkyl, t-butyl, isopropoxy, CF₃, OCF₃, SF₅, SF₃, halo, methylsulfone, alkoxy, amine, or nitrile;

wherein each Y in the B ring is independently CH, CR₈, or N; wherein at most two Ys in the B ring are N; each R₈ is independently H, alkyl, t-butyl, isopropoxy, CF₃, OCF₃, SF₅, SF₃, halo, methylsulfone, alkoxy, amine, or nitrile; and

wherein any of the alkyl, t-butyl, alkoxy, isopropoxy in any R₇, R₈, or combination thereof is each independently substituted or unsubstituted, branched or unbranched, or any combination thereof.

For example, Q may have one of the formulas:

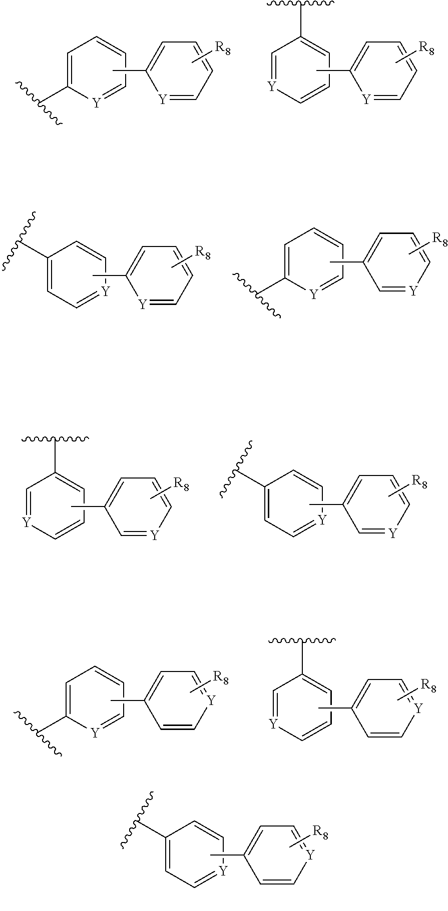

In any embodiment herein, Q may have the following formula:

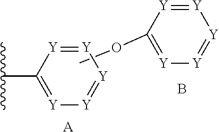

wherein each Y in the A ring is independently CH, CR₇, N, or C—(O—B ring); wherein at most two Ys in the A ring are N; wherein at most two Ys in the A ring are independently C—(O—B ring); each R₇ is independently H, D, alkyl, t-butyl, isopropoxy, CF₃, OCF₃, SF₅, SF₃, halo, methylsulfone, alkoxy, amine, or nitrile; and

wherein each Y in the B ring is independently CH, CR₈, or N; wherein at most two Ys in the B ring are N; each R₈ is independently H, alkyl, t-butyl, isopropoxy, CF₃, OCF₃, SF₅, SF₃, halo, methylsulfone, alkoxy, amine, or nitrile; and

wherein any of the alkyl, t-butyl, alkoxy, isopropoxy in any R₇, R₈, or combination thereof is each independently substituted or unsubstituted, branched or unbranched, or any combination thereof.

For example, Q may have one of the formulas:

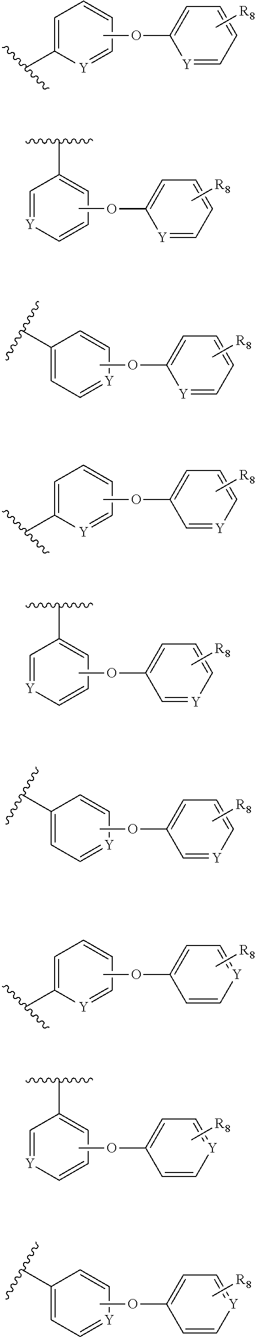

In any embodiment herein, Q may have the following formula:

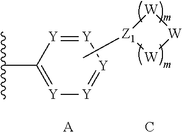

wherein each Y in the A ring is independently CH, CR₇, N, or C—(C ring); wherein at most two Ys in the A ring are N; wherein at most two Ys in the A ring are independently C—(C ring); each R₇ is independently H, D, alkyl, t-butyl, isopropoxy, CF₃, OCF₃, SF₅, SF₃, halo, methylsulfone, alkoxy, amine, or nitrile;

wherein Z₁ in the C ring is CH, N; each W in the C ring is independently CH₂, CHR₁₀, CR₁₀R₁₀, NH, NR₁₀, S, SO, SO₂, or O; each m in the C ring is independently 0-5; wherein the C ring is 3-12 members; each R₁₀ is independently H, alkyl, t-butyl, isopropoxy, CF₃, OCF₃, SF₅, SF₃, halo, F, methylsulfone, alkoxy, amine, or nitrile; and

wherein any of the alkyl, t-butyl, alkoxy, isopropoxy in any R₇, R₁₀, or combination thereof is each independently substituted or unsubstituted, branched or unbranched, or any combination thereof.

For example, Q may have one of the following formulas:

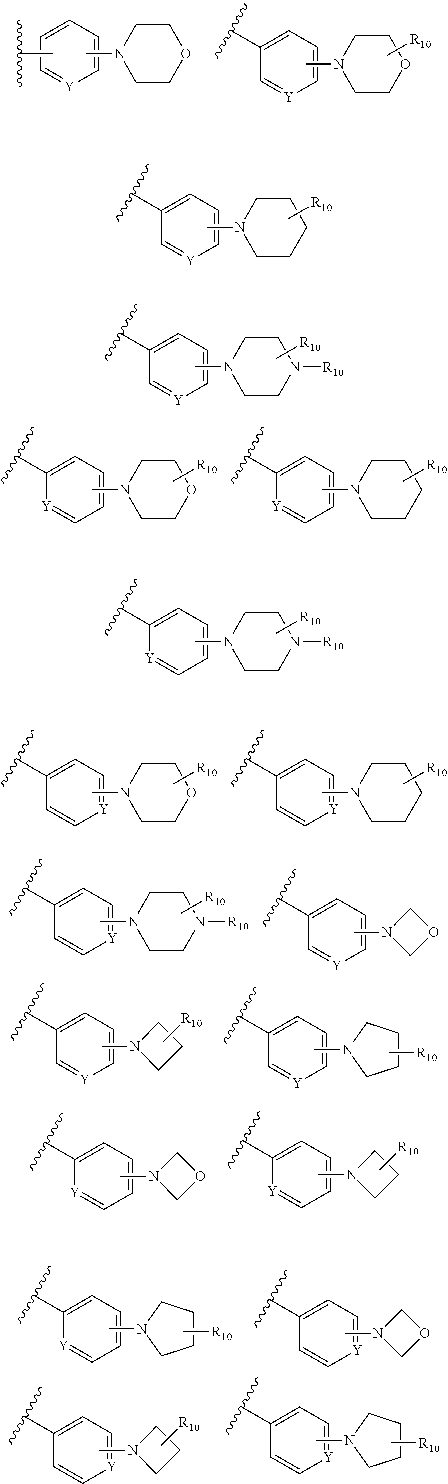

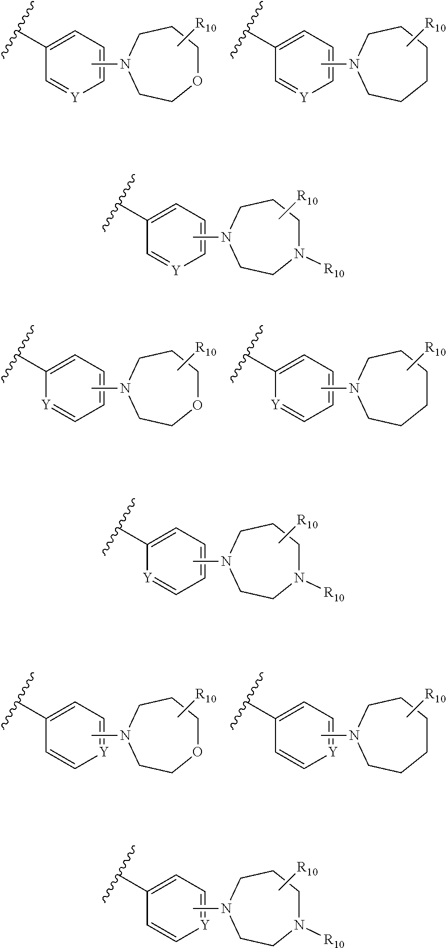

In any embodiment herein, Q may have the following formula:

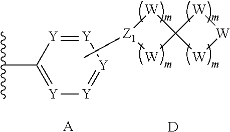

wherein each Y in the A ring is independently CH, CR₇, N, or C-(D ring); wherein at most two Ys in the A ring are N; wherein at most two Ys in the A ring are independently C-(D ring); each R₇ is independently H, D, alkyl, t-butyl, isopropoxy, CF₃, OCF₃, SF₅, SF₃, halo, methylsulfone, alkoxy, amine, or nitrile;

wherein Z₁ in the D ring is CH, N; each W in the D ring is independently CH₂, CHR₁₀, CR₁₀R₁₀, NH, NR₁₀, S, SO, SO₂, or O; each m in the D ring is independently 0-5; wherein the D ring is 7-23 members; each R₁₀ is independently H, alkyl, t-butyl, isopropoxy, CF₃, OCF₃, SF₅, SF₃, halo, F, methylsulfone, alkoxy, amine, or nitrile; and

wherein any of the alkyl, t-butyl, alkoxy, isopropoxy in any R₇, R₁₀, or combination thereof is each independently substituted or unsubstituted, branched or unbranched, or any combination thereof.

In any embodiment herein, Q may have the following formula:

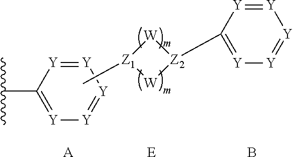

wherein each Y in the A ring is independently CH, CR₇, N, or C-(E-B ring); wherein at most two Ys in the A ring are N; wherein at most two Ys in the A ring are independently C-(E-B ring); each R₇ is independently H, D, alkyl, t-butyl, isopropoxy, CF₃, OCF₃, SF₅, SF₃, halo, methylsulfone, alkoxy, amine, or nitrile;

wherein Z₁ in the E ring is CH, N; wherein Z₂ in the E ring is CH, CR₉, or N; each R₉ is independently H, alkyl, t-butyl, isopropoxy, CF₃, OCF₃, SF₅, SF₃, halo, F, methylsulfone, alkoxy, amine, nitrile or hydroxy; each W in the E ring is independently CH₂, CHR₁₀, CR₁₀R₁₀, NH, NR₁₀, S, SO, SO₂, or O; each m in the E ring is independently 0-5; wherein the E ring is 3-12 members; each R₁₀ is independently H, alkyl, t-butyl, isopropoxy, CF₃, OCF₃, SF₅, SF₃, halo, F, methylsulfone, alkoxy, amine, or nitrile;

wherein each Y in the B ring is independently CH, CR₈, or N; wherein at most two Ys in the B ring are N; each R₈ is independently H, alkyl, t-butyl, isopropoxy, CF₃, OCF₃, SF₅, SF₃, halo, methylsulfone, alkoxy, amine, or nitrile; and

wherein any of the alkyl, t-butyl, alkoxy, isopropoxy in any R₇, R₈, R₉, R₁₀, or combination thereof is each independently substituted or unsubstituted, branched or unbranched, or any combination thereof.

For example, Q may have one of the formulas:

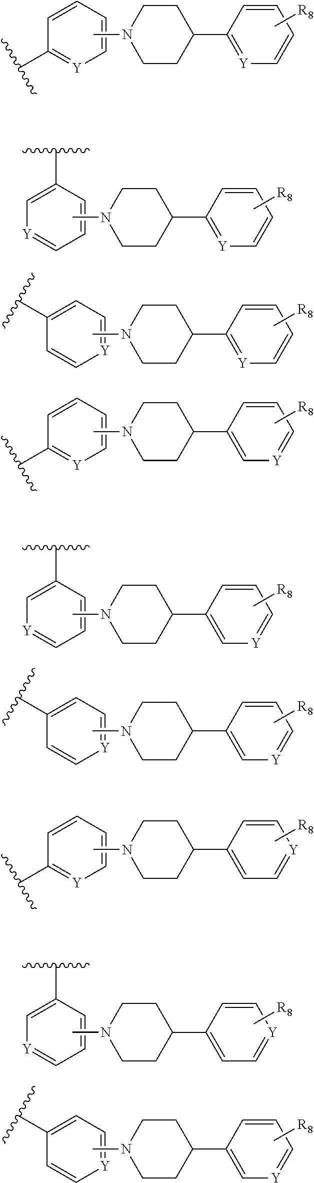

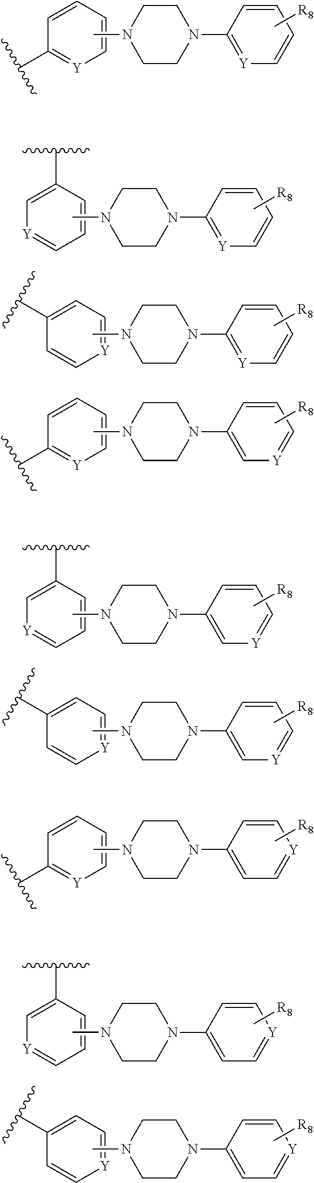

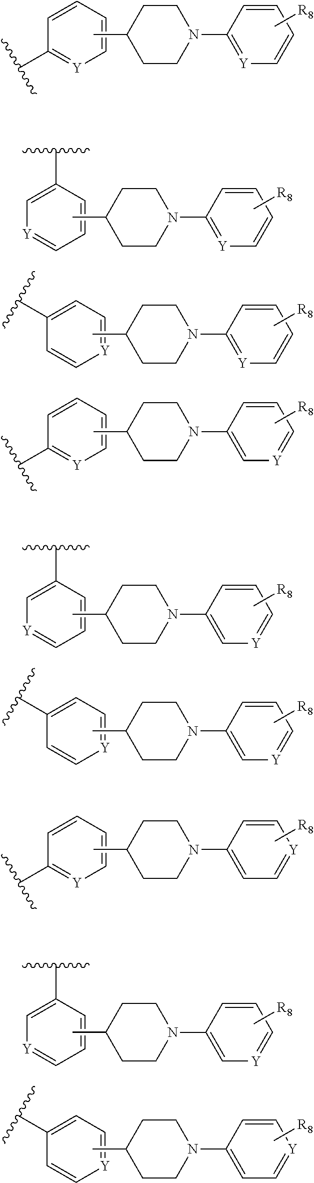

In any embodiment herein, Q may have the following formula:

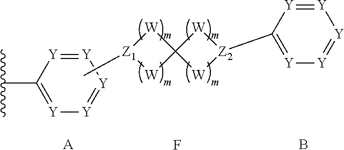

wherein each Y in the A ring is independently CH, CR₇, N, or C—(F—B ring); wherein at most two Ys in the A ring are N; wherein at most two Ys in the A ring are independently C—(F—B ring); each R₇ is independently H, D, alkyl, t-butyl, isopropoxy, CF₃, OCF₃, SF₅, SF₃, halo, methylsulfone, alkoxy, amine, or nitrile;

wherein Z₁ in the F ring is CH, N; wherein Z₂ in the F ring is CH, CR₉, or N; each R₉ is independently H, alkyl, t-butyl, isopropoxy, CF₃, OCF₃, SF₅, SF₃, halo, F, methylsulfone, alkoxy, amine, or nitrile; each W in the F ring is independently CH₂, CHR₁₀, CR₁₀R₁₀, NH, NR₁₀, S, SO, SO₂, or O; each m in the F ring is independently 0-5; wherein the F ring is 7-23 members; each R₁₀ is independently H, alkyl, t-butyl, isopropoxy, CF₃, OCF₃, SF₅, SF₃, halo, F, methylsulfone, alkoxy, amine, or nitrile;

wherein each Y in the B ring is independently CH, CR₈, or N; wherein at most two Ys in the B ring are N; each R₈ is independently H, alkyl, t-butyl, isopropoxy, CF₃, OCF₃, SF₅, SF₃, halo, methylsulfone, alkoxy, amine, or nitrile; and

wherein any of the alkyl, t-butyl, alkoxy, isopropoxy in any R₇, R₈, R₉, R₁₀, or combination thereof is each independently substituted or unsubstituted, branched or unbranched, or any combination thereof.

For example, Q may have one of the formulas:

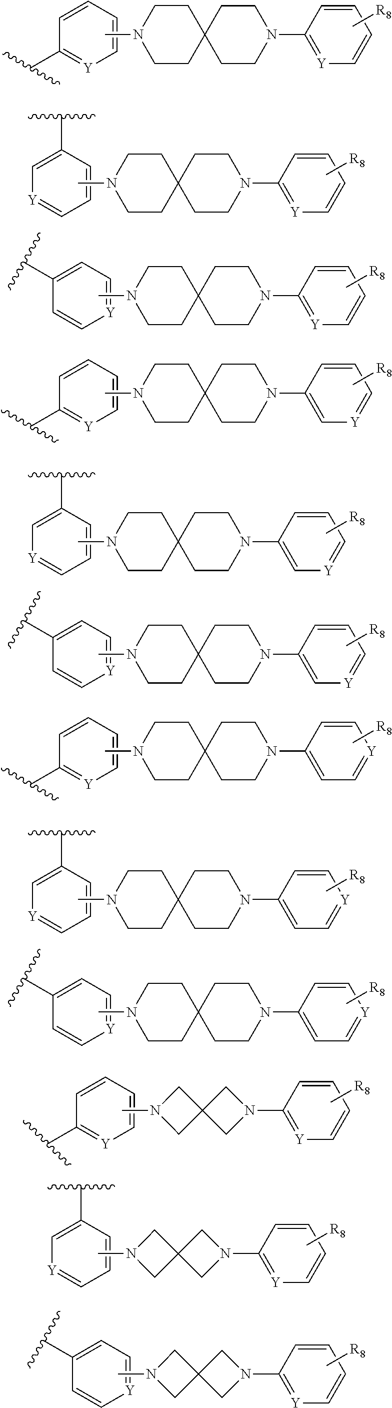

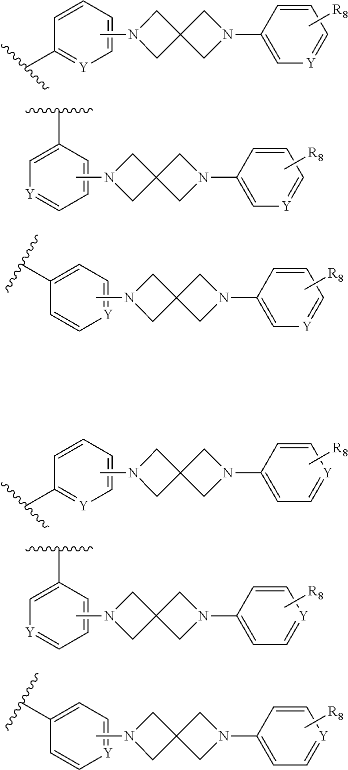

In any embodiment herein, Q may have the following formula:

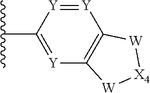

wherein X₄ is CH₂, CHR₈, CR₈R₈, S, SO, SO₂, O, NH, NR₈,

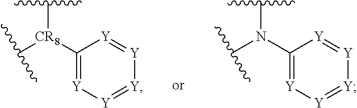

wherein each R₈ is independently H, alkyl, t-butyl, isopropoxy, CF₃, OCF₃, SF₅, SF₃, halo, methylsulfone, alkoxy, amine, or nitrile;

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile;

wherein each W is independently CH₂, CHR₁₀, CR₁₀R₁₀, NH, NR₁₀, S, SO, SO₂, or 0;

each R₁₀ is independently H, alkyl, t-butyl, isopropoxy, CF₃, OCF₃, SF₅, SF₃, halo, F, methylsulfone, alkoxy, amine, or nitrile;

and wherein any of the alkyl, t-butyl, alkoxy, isopropoxy in any R group is each independently substituted or unsubstituted, branched or unbranched, or any combination thereof.

For example, Q may have one of the following formulas:

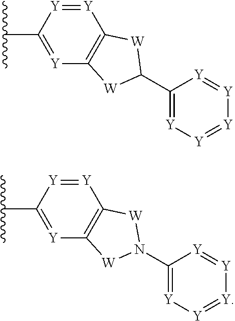

In any embodiment herein, Q may have one of the following formulas:

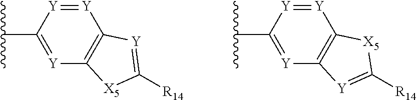

wherein X₅ is CH₂, CHR₈, CR₈R₈, S, SO, SO₂, O, NH, NR₈; wherein each R₈ is independently H, alkyl, t-butyl, isopropoxy, CF₃, OCF₃, SF₅, SF₃, halo, methylsulfone, alkoxy, amine, or nitrile;

wherein R₁₀ is independently H, alkyl, t-butyl, isopropoxy, CF₃, OCF₃, SF₅, SF₃, halo, F, methylsulfone, alkoxy, amine, nitrile, or

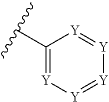

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile;

and wherein any of the alkyl, t-butyl, alkoxy, isopropoxy in any R group is each independently substituted or unsubstituted, branched or unbranched, or any combination thereof.

For example, Q may have one of the following formulas:

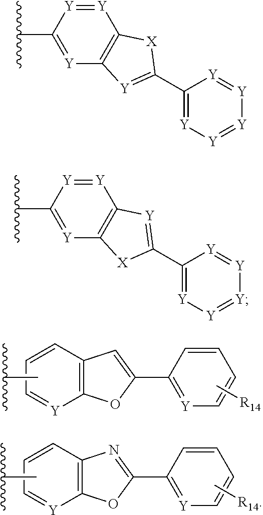

In any embodiment herein, Q may have the following formula:

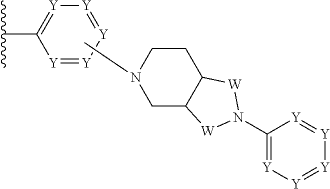

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile;

wherein each W is independently CH₂, CHR₁₀, CR₁₀R₁₀, NH, NR₁₀, S, SO, SO₂, or O; each R₁₀ is independently H, alkyl, t-butyl, isopropoxy, CF₃, OCF₃, SF₅, SF₃, halo, F, methylsulfone, alkoxy, amine, or nitrile;

and wherein any of the alkyl, t-butyl, alkoxy, isopropoxy in any R group is each independently substituted or unsubstituted, branched or unbranched, or any combination thereof.

In any embodiment herein, Q may have one of the following formulas:

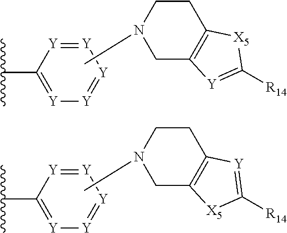

wherein X₅ is CH₂, CHR₈, CR₈R₈, S, SO, SO₂, O, NH, NR₈; wherein each R₈ is independently H, alkyl, t-butyl, isopropoxy, CF₃, OCF₃, SF₅, SF₃, halo, methylsulfone, alkoxy, amine, or nitrile;

wherein R₁₄ is independently H, alkyl, t-butyl, isopropoxy, CF₃, OCF₃, SF₅, SF₃, halo, F, methylsulfone, alkoxy, amine, nitrile, or

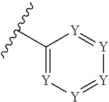

and

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile;

and wherein any of the alkyl, t-butyl, alkoxy, isopropoxy in any R group is each independently substituted or unsubstituted, branched or unbranched, or any combination thereof.

For example, Q may have one of the following formulas:

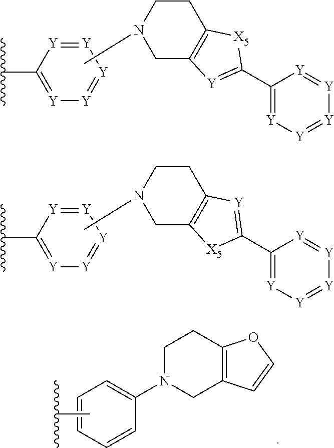

In any embodiment herein, Q may have one of the following formulas:

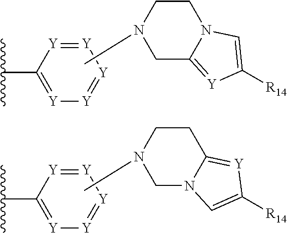

wherein R₁₀ is independently H, alkyl, t-butyl, isopropoxy, CF₃, OCF₃, SF₅, SF₃, halo, F, methylsulfone, alkoxy, amine, nitrile, or

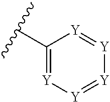

and

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile;

and wherein any of the alkyl, t-butyl, alkoxy, isopropoxy in any R group is each independently substituted or unsubstituted, branched or unbranched, or any combination thereof.

For example, Q may have one of the following formulas:

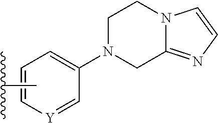

In any embodiment herein, Q may have the following formulas:

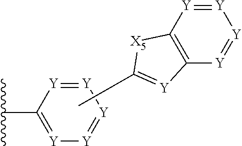

wherein X₅ is CH₂, CHR₈, CR₈R₈, S, SO, SO₂, O, NH, NR₈; wherein each R₈ is independently H, alkyl, t-butyl, isopropoxy, CF₃, OCF₃, SF₅, SF₃, halo, methylsulfone, alkoxy, amine, or nitrile; and

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile.

For example, Q may have the following formula:

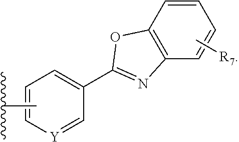

In any embodiment herein, Q may have one of the following formulas:

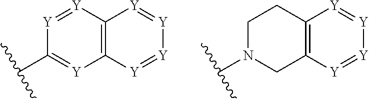

wherein each Y is independently CH, CR₇, or N; wherein at most two Ys in any one ring are N; each R₇ is independently H, D, alkyl, t-butyl, isopropoxy, CF₃, OCF₃, SF₅, SF₃, halo, methylsulfone, alkoxy, amine, or nitrile.

In any embodiment herein, Q may be an alkynyl, propargyl, triazole or substituted triazole group, or PEGylated derivative thereof, such as, for example, Q-groups which are suitable in or the product of the well-known “click” chemistry reactions. For example, Q may have any of the following formulas:

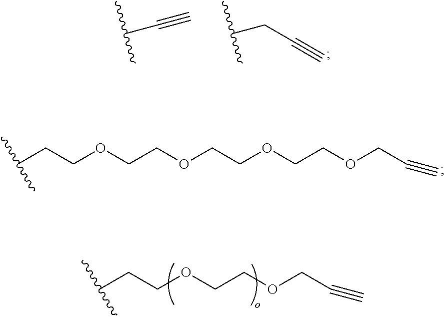

wherein o is 1-30;

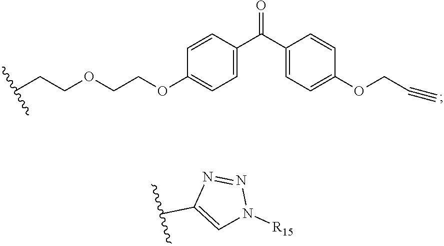

wherein R₁₅ may be H, C₁₋₆ alkyl, C₃₋₈ cycloalkyl, C₆₋₁₂ aryl, or C₅₋₆ heteroaryl.

One example of a click reaction includes:

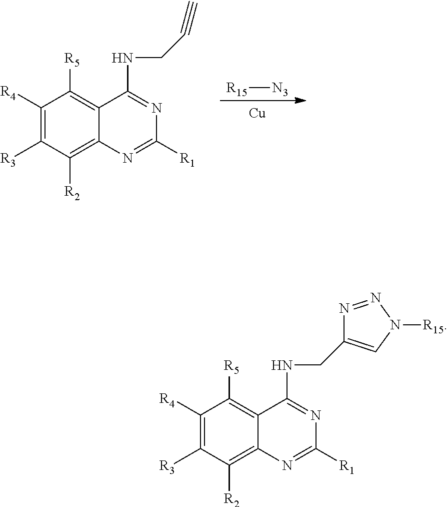

Other exemplary Cyt-bc1:aa3 inhibitors, F₁F₀-ATP synthase inhibitors, and NADH dehydrogenase (NDH-2) activator are shown below:

Cyt:bc1:aa3 inhibitors:

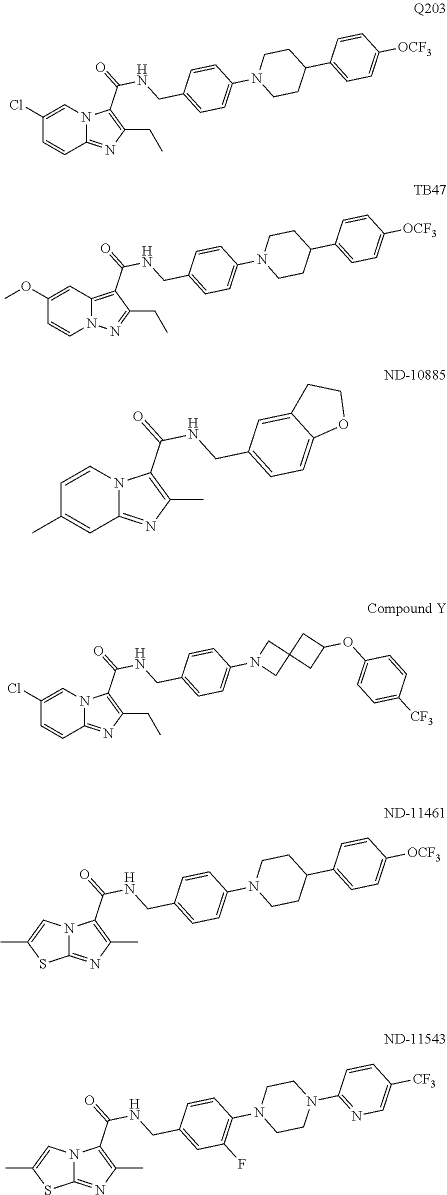 [SMILES: CCc1nc2ccc(Cl)cn2c1C(=O)NCc1ccc(N2CCC(c3ccc(OC(F)(F)F)cc3)CC2)cc1 ; CCc1nn2ccc(OC)cc2c1C(=O)NCc1ccc(N2CCC(c3ccc(OC(F)(F)F)cc3)CC2)cc1 ; Cc1ccn2c(C(=O)NCc3ccc4c(c3)CCO4)c(C)nc2c1 ; CCc1nc2ccc(Cl)cn2c1C(=O)NCc1ccc(N2CC3(CC(Oc4ccc(C(F)(F)F)cc4)C3)C2)cc1 ; Cc1cn2c(C(=O)NCc3ccc(N4CCC(c5ccc(OC(F)(F)F)cc5)CC4)cc3)c(C)nc2s1 ; Cc1cn2c(C(=O)NCc3ccc(N4CCN(c5ccc(C(F)(F)F)cn5)CC4)c(F)c3)c(C)nc2s1]

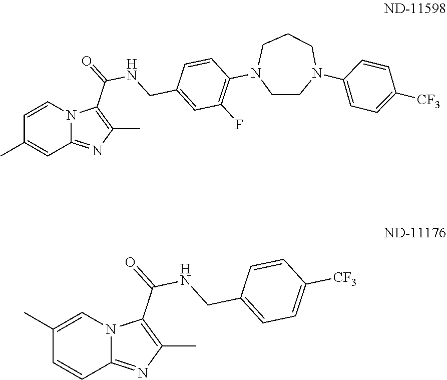 [SMILES: Cc1ccn2c(C(=O)NCc3ccc(N4CCCN(c5ccc(C(F)(F)F)cc5)CC4)c(F)c3)c(C)nc2c1 ; Cc1ccc2nc(C)c(C(=O)NCc3ccc(C(F)(F)F)cc3)n2c1]

FOF1-ATP Synthase Inhibors:

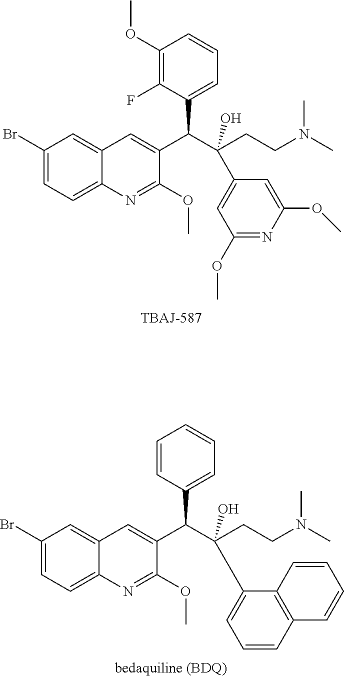 [SMILES: COc1cc([C@](O)(CCN(C)C)[C@@H](c2cc3cc(Br)ccc3nc2OC)c2cccc(OC)c2F)cc(OC)n1 ; COc1nc2ccc(Br)cc2cc1[C@@H](c1ccccc1)[C@@](O)(CCN(C)C)c1cccc2ccccc12]

NADH Dehydrogenase (NDH-2) Activator:

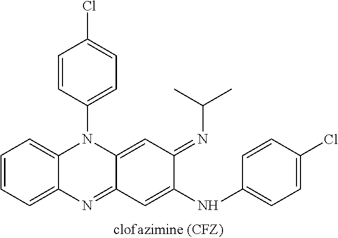 [SMILES: CC(C)/N=c1\cc2n(-c3ccc(Cl)cc3)c3ccccc3nc-2cc1Nc1ccc(Cl)cc1]

Other antibacterial agents, which may be used in combination with the Cyt-bd inhibitors described herein, include Rifampicin (RIF), Pyrazinamide (PZA), Isoniazid (INH), and Clarithromycin (CLA).

General

The following general discussion is provided for purposes of better understanding and is not intended to be limiting unless otherwise specified.

An alkyl group is suitably a univalent, acyclic, straight or branched, substituted or unsubstituted, saturated or unsaturated, hydrocarbon radical. The alkyl group may have the general formula (notwithstanding optional substitution or the like) —CₙH₂ₙ₊₁. In some embodiments, n is 1-6 ((C₁-C₆) alkyl), which may suitably include C₁, C₂, C₃, C₄, C₅, and C₆ alkyl groups. The alkyl group may be straight or branched, substituted or unsubstituted, saturated or unsaturated, or any combination thereof. One or more hydrogens may be optionally and independently replaced by one or more substituent groups. One or more carbon atoms may be optionally and independently replaced with one or more heteroatoms such as O, S, N, B, or any combination thereof. In some embodiments, the alkyl group is attached to the parent structure through one or more independent divalent intervening substituent groups. Some examples of alkyl groups, which are not intended to be limiting, include methyl, ethyl, n-propyl, isopropyl, n-butyl, iso-butyl, secondary-butyl, tertiary-butyl, and the like. An alkyl group may suitably be a univalent, acyclic, straight or branched, substituted or unsubstituted saturated C₁-C₆, C₁-C₅, C₁-C₄, C₁-C₃, or C₁-C₂ hydrocarbon radical. An alkyl group may suitably be a univalent, straight, substituted or unsubstituted, saturated C₁-C₆, C₁-C₅, C₁-C₄, C₁-C₃, or C₁-C₂ hydrocarbon radical. An alkyl group may suitably be a univalent, straight, unsubstituted, saturated C₁-C₆, C₁-C₅, C₁-C₄, C₁-C₃, or C₁-C₂ hydrocarbon radical.

One or more than one of the hydrogens on an alkyl group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

An alkenyl group is suitably a univalent, straight or branched, substituted or unsubstituted, unsaturated hydrocarbon radical. The alkenyl group may have the general formula (notwithstanding optional substitution, higher degree of unsaturation, or the like) —CₙH₂ₙ₋₂. In some embodiments, n is 2-22 ((C₂-C₂₂) alkenyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, and C₂₂ alkenyl groups. The alkenyl group may be straight or branched, substituted or unsubstituted, have more than one degree of unsaturation, or any combination thereof. One or more hydrogens may be optionally and independently replaced by one or more substituent groups. One or more carbon atoms may be optionally and independently replaced with one or more heteroatoms such as O, S, N, B, or any combination thereof. In some embodiments, the alkenyl group is attached to the parent structure through one or more independent divalent intervening substituent groups. Some examples of alkenyl groups, which are not intended to be limiting, include ethenyl, 1-propenyl, 2-propenyl (allyl), iso-propenyl, 2-methyl-1-propenyl, 1-butenyl, 2-butenyl, alkadienes, alkatrienes, terpenes, and the like. An alkenyl group may suitably be a univalent, straight or branched, substituted or unsubstituted, unsaturated C₂-C₂₂, C₂-C₁₅, C₂-C₁₀, C₂-C₈, or C₂-C₆ hydrocarbon radical. An alkenyl group may suitably be a univalent, straight or branched, unsubstituted, unsaturated C₂-C₂₂, C₂-C₁₅, C₂-C₁₀, C₂-C₈, or C₂-C₆ hydrocarbon radical. An alkenyl group may suitably be a univalent, straight, unsubstituted, unsaturated C₂-C₂₂, C₂-C₁₅, C₂-C₁₀, C₂-C₈, or C₂-C₆ hydrocarbon radical. An alkenyl group may suitably be a univalent, straight, unsubstituted, unsaturated C₂-C₆ hydrocarbon radical. An alkenyl group may suitably be a terpene, such as geranyl, farnesyl, geranylgeranyl, or the like.

One or more than one of the hydrogens on an alkenyl group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, aryl, heteroaryl, cyclic amine, cycloalkyl, heterocycle, or combination thereof.

A cycloalkyl group is suitably a univalent, mono- or polycyclic, substituted or unsubstituted, saturated or unsaturated hydrocarbon radical. The cycloalkyl group may have the general formula (notwithstanding optional unsaturation, substitution, or the like) —CₙH₂ₙ₋₁. In some embodiments, n is 3-8 ((C₃-C₈) cycloalkyl), which may suitably include C₃, C₄, C₅, C₆, C₇, or C₈ cycloalkyl groups. As mentioned, the cycloalkyl group may be substituted or unsubstituted, saturated or unsaturated, mono-, bi-, tri-, or poly-cyclic, or any combination thereof. One or more hydrogens may be optionally and independently replaced by one or more substituent groups. One or more carbon atoms may be optionally and independently replaced with one or more heteroatoms such as O, S, N, B, or any combination thereof. In some embodiments, the cycloalkyl group is attached to the parent structure through one or more independent divalent intervening substituent groups. Some examples of cycloalkyl groups, which are not intended to be limiting, include cyclopropyl, cyclobutyl, cyclopentyl, cyclohexyl, cycloheptyl, cyclooctyl, and the like. In the case of polycyclic groups, one or more of the rings may be tethered together via bond or other divalent intervening substituent group, fused (e.g., in which one or more rings shares two or more carbon atoms or heteroatoms, joined via a single atom (e.g., spiro compound), or bridged. A cycloalkyl group may suitably be a univalent, mono- or polycyclic, substituted or unsubstituted, saturated or unsaturated C₃-C₈, C₃-C₆, C₃-C₅, or C₃-C₄ hydrocarbon radical. A cycloalkyl group may suitably be a univalent, mono-cyclic, substituted or unsubstituted, saturated C₃-C₈, C₃-C₆, C₃-C₅, or C₃-C₄ hydrocarbon radical. A cycloalkyl group may suitably be a univalent, mono-cyclic, unsubstituted, saturated C₃-C₈, C₃-C₆, C₃-C₅, or C₃-C₄ hydrocarbon radical.

One or more than one of the hydrogens on a cycloalkyl group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

An alkoxy group is suitably a univalent radical derived from an —O-alkyl group, which may suitably include C₁, C₂, C₃, C₄, C₆, and C₆ —O-alkyl groups. In some embodiments, the alkoxy group may be attached to the parent structure through one or more independent divalent intervening substituent groups. An alkoxy group may suitably be a univalent, acyclic, straight or branched, substituted or unsubstituted saturated C₁-C₆, C₁-C₅, C₁-C₄, C₁-C₃, or C₁-C₂ alkoxy radical. An alkoxy group may suitably be a univalent, straight, substituted or unsubstituted, saturated C₁-C₆, C₁-C₅, C₁-C₄, C₁-C₃, or C₁-C₂ alkoxy radical. An alkoxy group may suitably be a univalent, straight, unsubstituted, saturated C₁-C₆, C₁-C₅, C₁-C₄, C₁-C₃, or C₁-C₂ alkoxy radical.

One or more than one of the hydrogens on an alkoxy group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

A cyclic amine is suitably a univalent radical derived from a cycloalkyl group in which one or more of the ring carbons is replaced with a nitrogen. A cyclic amine may suitably have 3-9 ring members, in which one or more nitrogens form a ring with 2-8 carbons. For example, a cyclic amine may have a formula such as:

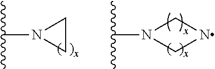

wherein each x may independently be 1-8, and, in the case of the second structure, the “N.” radical may be satisfied with a hydrogen or other substituent. Each x may independently be 1, 2, 3, 4, 5, 6, 7, or 8. The cyclic amine may have 3, 4, 5, 6, 7, 8, or 9 members, including the nitrogen and if present other heteroatom. In the cyclic amine, one or more than one of the further ring carbons may be suitably replaced with one or more further heteroatoms, e.g., N, O, P, S, oxidized form thereof, or combination thereof. In the cyclic amine, one or more than one of the further ring carbons may be suitably replaced with one or more N, O, or combination thereof. The cyclic amine may be suitably substituted or unsubstituted, saturated or unsaturated, mono- or poly-cyclic, or any combination thereof. One or more hydrogens may be suitably replaced by one or more substituent groups. The cyclic amine may suitably attached to the parent structure through a ring carbon or a nitrogen. In some embodiments, the cyclic amine may be attached to the parent structure through one or more independent divalent intervening substituent groups. Non-limiting examples of cyclic amines include aziridine, azetidine, morpholine, thiomorpholine, piperidine, piperazine, and the like.

One or more than one of the hydrogens on a cyclic amine group may be independently replaced with a C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

A heterocyclic group is suitably a univalent, substituted or unsubstituted, saturated or unsaturated, mono- or polycyclic hydrocarbon radical that contains one or more heteroatoms in one or more of the rings. The heterocyclic group may suitably be a C₃-C₂₀ cyclic group, in which one or more ring carbons is independently replaced with one or more heteroatoms. The C₃-C₂₀ heterocyclic group may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cyclic groups, or any combination thereof, in which one or more ring carbons is independently replaced with one or more heteroatoms. The heteroatom or heteroatoms may be suitably selected from one or more of N, O, or S, or any combination thereof. The N or S or both may be independently substituted with one or more substituents. The N or S or both may be independently substituted with hydrogen or other substituent. The heterocyclic group may be substituted or unsubstituted, saturated or unsaturated, mono-, bi-, tri-, or poly-cyclic, or any combination thereof. One or more hydrogens in the heterocyclic group may be optionally and independently replaced by one or more substituent groups. If desired, the heterocyclic group may include one or more carbon-carbon double bonds, carbon-carbon triple bonds, carbon-nitrogen double bonds, or any combination thereof. The heterocyclic group may suitably attached to the parent structure through a ring carbon or heteroatom, for example nitrogen. In some embodiments, the heterocyclic group is attached to the parent structure through one or more independent divalent intervening substituent groups. Some examples of heterocyclic groups, which are not intended to be limiting, include cyclic amine, azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl, and the like. The heterocyclic group may suitably be a C₃-C₁₂, C₃-C₁₀, C₃-C₈, or C₃-C₆, saturated cyclic group, in which one or more ring carbons is independently replaced with one or more N, O, or other heteroatom. The heterocyclic group may suitably be a substituted or unsubstituted C₅-C₁₂ aryl group, in which at most two of the ring carbons are replaced with a nitrogen heteroatom. The heteroaryl group may suitably be a substituted or unsubstituted C₅-C₁₂ aryl group, in which one of the ring carbons are replaced with a nitrogen heteroatom.

One or more than one of the hydrogens on a heterocyclic group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

An aryl group is suitably a univalent, substituted or unsubstituted, monocyclic or polycyclic aromatic hydrocarbon radical. An aryl group may be a radical which, in accordance with Hickeys threory, includes a cyclic, delocalized (4n+2) pi-electron system. The aryl group may suitably be a C₆-C₁₂ aryl group, which range includes C₆, C₇, C₈, C₉, C₁₀, C₁₁, and C₁₂ aryl groups. The aryl group may be substituted or unsubstituted, be substituted with two or more groups that taken together form a cyclic group, or any combination thereof. In some embodiments, the aryl group is attached to the parent structure through one or more independent divalent intervening substituent groups. Some examples of aryl groups, which are not intended to be limiting, include phenyl, naphthyl, tetrahydronaphthyl, and the like.

One or more than one of the hydrogens on an aryl group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

A heteroaryl group is suitably a univalent, substituted or unsubstituted, monocyclic or polycyclic aromatic hydrocarbon radical in which one or more ring carbons is independently replaced with one or more heteroatoms selected from O, S and N. In addition to said heteroatom, the heteroaryl group may optionally have up to 1, 2, 3, or 4 nitrogen atoms in the ring. The heteroaryl group is an aryl group in which one or more ring carbons are independently replaced with one or more heteroatoms. A heteroaryl group is suitably an aromatic radical, which contains one or more heteroatoms and which, in accordance with Hickeys threory, includes a cyclic, delocalized (4n+2) pi-electron system. The heteroaryl group may suitably be a C₅-C₂₀ heteroaryl group. The C₅-C₂₀ heteroaryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups, or any combination thereof in which one or more than one ring carbon is independently replaced with one or more heteroatoms. The heteroaryl group may be substituted or unsubstituted, be substituted with two or more groups that taken together form a cyclic group, or any combination thereof. The heteroaryl group may be suitably attached to the parent structure through a ring carbon or heteroatom, or through one or more independent divalent intervening substituent groups. Some examples of heteroaryl groups, which are not intended to be limiting, include heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like. The heteroaryl group may suitably be a substituted or unsubstituted C₆-C₁₂ aryl group, in which at most two of the ring carbons are replaced with a nitrogen heteroatom. The heteroaryl group may suitably be a substituted or unsubstituted C₆-C₁₂ aryl group, in which one of the ring carbons are replaced with a nitrogen heteroatom.

One or more than one of the hydrogens on a heteroaryl group may be independently replaced with a C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

In embodiments, suitable examples of the heteroaryl or heterocycles include furan, substituted, furan, thiophene, substituted thiophene, oxazole, substituted oxazole, isoxazole, substituted isoxazole, imidazole, substituted imidazole, pyrrole, substituted pyrrole, pyrrolidine, substituted pyrrolidine, tetrahydrofuran, substituted tetrahydrofuran, pyridine, substituted pyridine, piperidine, substituted piperidine, pyrimidine, substituted pyrimidine, pyrazine, substituted pyrazine, azepine, substituted azepine, 1,4-diazepine, substituted 1,4-diazepine, 4H-pyran, substituted 4H-pyran, tetrahydropyran, substituted tetrahydropyran, indole, substituted indole, quinoline, substituted quinoline, isoquinoline, substituted isoquionoine, chroman, substituted chroman, purine, substituted purine, pteridine, substituted pteridine, benzimidazole, substituted benzimidazole, benzothiazole, substituted benzothiazole, benzoxazole, substituted benzoxazole, benzofuran, substituted benzofuran, benzothiazole, substituted benzothiazole, 1H-indazole, and substituted 1H-indazole. Some examples of the substituents for these substituted heteroaryls or substituted heterocycles include one or more of C₁-C₆ alkyl, alkenyl, alkoxy, t-butyl, isopropoxy, amine, nitrile, halogen, F, Cl, hydroxy, CF₃, OCF₃, SF₃, SF₅, sulfate, methylsulfone, nitrate, carboxylate, carboxylic acid, aryl, heteroaryl, cyclic amine, cycloalkyl, heterocycle, or combination thereof.

A bi-aryl group is suitably two aryl groups bound together either directly via covalent bond, or through an intervening methylene, ethylene, propylene, or the like. The aryl groups may be the same or different. The bi-aryl group may suitably be directly connected to the parent structure through one of the ring carbons of one of the aryl groups, or through an intervening divalent substituent. The bi-aryl group may suitably be a —C₆H₄—C₆H₅.

One or more than one of the hydrogens on a bi-aryl group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

A bi-aryl ether group is suitably two aryl groups bound together via an intervening oxygen. The aryl groups may be the same or different. The bi-aryl ether group may suitably be directly connected to the parent structure through one of the ring carbons of one of the aryl groups, or through an intervening divalent substituent. The bi-aryl group may suitably be a —C₆H₄—O—C₆H₅.

One or more than one of the hydrogens on a bi-aryl ether group may be independently replaced with a C₁-C₆ alkyl, alkenyl, alkoxy, t-butyl, isopropoxy, amine, nitrile, halogen, F, C₁, hydroxy, CF₃, OCF₃, SF₃, SF₅, sulfate, methylsulfone, nitrate, carboxylate, carboxylic acid, carboxamide, aryl, heteroaryl, cyclic amine, cycloalkyl, heterocycle, or combination thereof.

A halo group is suitably a univalent halogen radical or halogen-containing substituent group, e.g., one that is or contains one or more F, Br, Cl, I, or combination thereof. The halo may suitably be F or Cl. In some embodiments, the halo is suitably F.

The pharmaceutically acceptable carrier is not particularly limiting, and it may be selected from known or common solvents, diluents, dispersions, powders, water, saline, DMSO, ethanol, and the like, which are easily determined by one of ordinary skill in the subject art given the compounds and subjects disclosed herein.

### EXAMPLES

The examples below are provided for a better understanding and are not intended to be limiting.

Preparation of bd oxidase inhibitors can be carried out by classical S_{N}AR, methods described in J. Med. Chem., 1999, 42 (22), pp 4705-4713 and shown below:

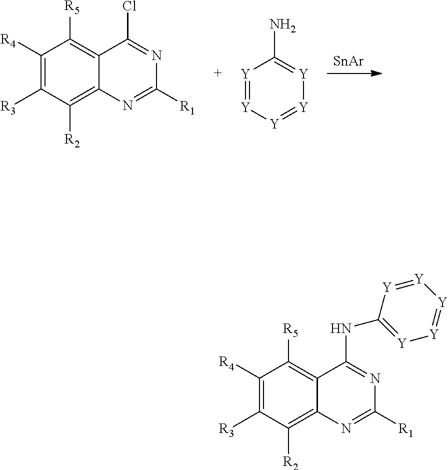

Specific S_{N}AR examples:

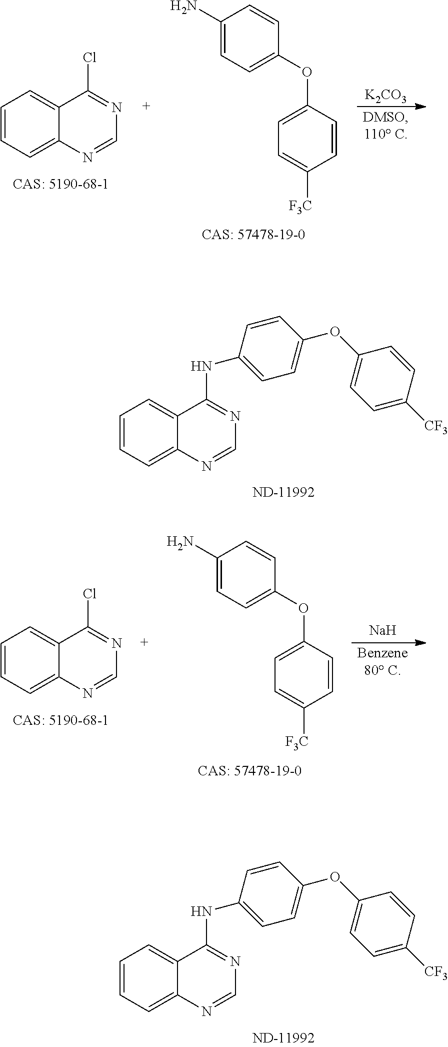

By Pallidum cross coupling (Buchwald) reaction:

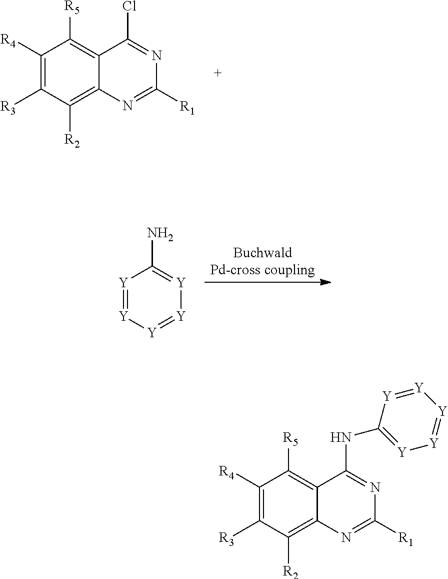

### Specific Example

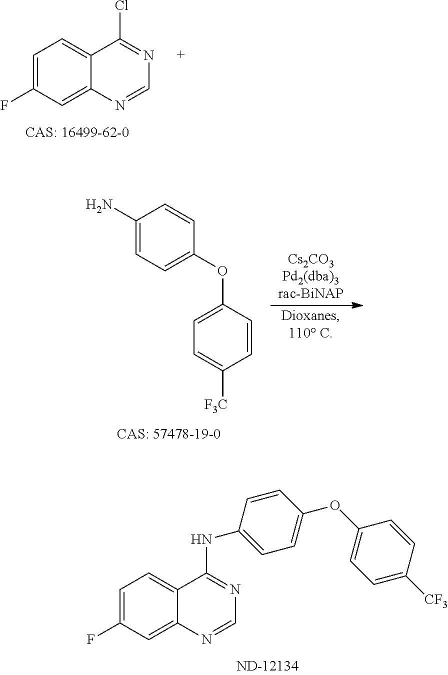 [SMILES: Fc1ccc2c(Cl)ncnc2c1 ; Nc1ccc(Oc2ccc(C(F)(F)F)cc2)cc1 ; "Cs2CO3Pd2(dba)3rac-BiNAP" ; Fc1ccc2c(Nc3ccc(Oc4ccc(C(F)(F)F)cc4)cc3)ncnc2c1]

By Phosphonium coupling reaction as described by Fu-An Kang in Progress in Heterocyclic Chemistry, Volume 27, 2015, 29-59:

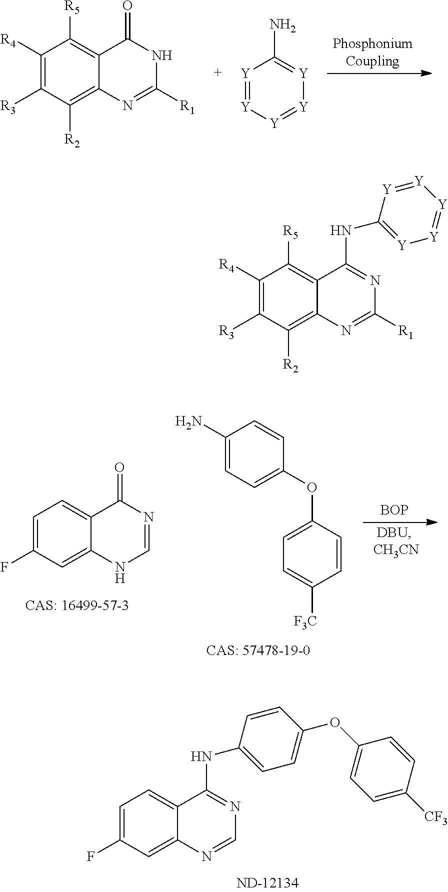

Preparation of Select Quinazoline Intermediates:

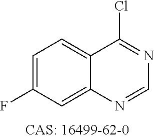 [SMILES: Fc1ccc2c(Cl)ncnc2c1]

4-Chloro-7-fluoroquinazoline (CAS: 16499-62-0) can be prepared by the methods in literature for example Bioorganic & Medicinal Chemistry Letters (2017), 27(21), 4885-4888; MedChemComm (2014), 5(9), 1290-1296; PCT Int. Appl., 2007071963, 28 Jun. 2007; U.S. Pat. Appl. Publ., 20050187231, 25 Aug. 2005.

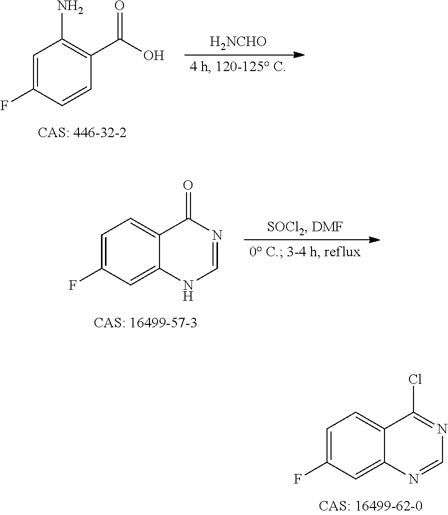

A General/Typical Procedure includes: A solution of 18.2 g of CAS: 446-32-2 (100 mmol) in 76.5 g (64 ml) of formamide (1.7 mol) was heated under reflux for 4 hrs at 120-125° C. Solvent was removed under reduced pressure and the crude solid was recrystallized from ethyl alcohol to give 12.7 g of compound CAS: 16499-57-3 (yield, 87%). To 7.3 g of compound CAS: 16499-57-3 (50 mmol) was added dropwise 230 ml of thionyl chloride (2 mol) at 0° C. with stirring. To the mixture was added 2-3 drops of N,N-dimethylformamide and the reaction heated under reflux for 3-4 hrs. Thionyl chloride was removed under reduced pressure and the resulting residue was washed with sodium carbonate. The product was extracted with ethyl acetate and the organic layer was dried over MgSO₄, filtered and concentrated under reduced pressure. The crude product was purified by SiO₂ column chromatography to give 4-chloro-7-fluoroquinazoline (CAS: 16499-62-0).

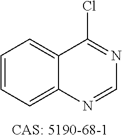 [SMILES: Clc1ncnc2ccccc12]

4-Chloroquinazoline (CAS: 5190-68-1) can be prepared by the methods in the literature, for example Bioorganic & Medicinal Chemistry Letters, 27(21), 4885-4888; 2017; RSC Advances, 7(54), 34005-34011; 2017; Journal of Heterocyclic Chemistry, 54(4), 2548-2555; 2017; Beilstein Journal of Organic Chemistry, 13, 174-181; 2017; PCT Int. Appl., 2016146074, 22 Sep. 2016.

### Synthesis of Bi-Aryl Ether Aniline

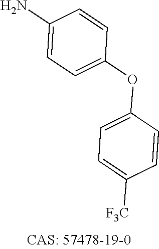 [SMILES: Nc1ccc(Oc2ccc(C(F)(F)F)cc2)cc1]

4-(4-(Trifluoromethyl)phenoxy)aniline (CAS: 57478-19-0) can be prepared by the methods within literature including the Journal of Medicinal Chemistry, 60(13), 5392-5406; 2017; MedChemComm, 6(4), 671-676; 2015; Journal of Medicinal Chemistry, 56(11), 4811-4815; 2013.

### Synthesis of N-(4-(4-(trifluoromethyl)phenoxy)phenyl)quinazolin-4-amine

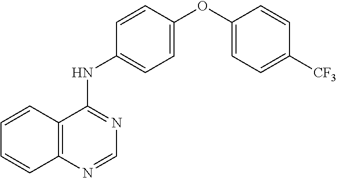 [SMILES: FC(F)(F)c1ccc(Oc2ccc(Nc3ncnc4ccccc34)cc2)cc1]

Synthesis of N-(4-(4-(trifluoromethyl)phenoxy)phenyl)quinazolin-4-amine. 4-Chloroquinazoline (CAS: 5190-68-1, 700 mg, 4.3 mmol), 4-(4-(trifluoromethyl)phenoxy)aniline (CAS: 57478-19-0, 1.08 g, 4.3 mmol) and potassium carbonate (587 mg, 4.3 mmol) were dissolved in 15 mL of DMSO. The reaction was heated to 110° C. for 12 hours. The reaction was concentrated in vacuo and the residue was dissolved in CH₂C₁₂ and washed with 5% acetic acid solution (2×), water and brine. The organic phase was collected, dried over sodium sulfate (Na₂SO₄), filtered and then concentrated in vacuo. Crude material obtained was purified by silica gel column chromatography with a 50% ethyl acetate: CH₂C₁₂ solvent system to give 1.27 g (78%) of N-(4-(4-(trifluoromethyl)phenoxy)phenyl)quinazolin-4-amine. Recrystallization from boiling isopropanol or CH₃CN afforded lightly colored crystals. ¹H NMR (300 MHz, CDCl₃) δ 8.75 (s, 1H), 7.93 (dd, J=8.3, 1.3 Hz, 2H), 7.87-7.74 (m, 2H), 7.74 (s, 1H), 7.58 (dd, J=8.5, 7.1 Hz, 4H), 7.16-7.02 (m, 4H). ¹⁹F NMR (282 MHz, CDCl₃)) 6-61.72 (s, 3F).

### Synthesis of 7-fluoro-N-(4-(4-(trifluoromethyl)phenoxy)phenyl)quinazolin-4-amine

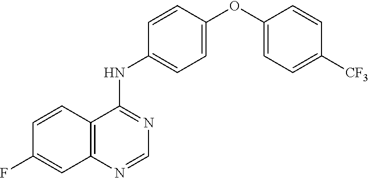 [SMILES: Fc1ccc2c(Nc3ccc(Oc4ccc(C(F)(F)F)cc4)cc3)ncnc2c1]

Synthesis of 7-fluoro-N-(4-(4-(trifluoromethyl)phenoxy)phenyl)quinazolin-4-amine. 4-Chloro-7-fluoroquinazoline (CAS: 16499-62-0, 480 mg, 2.6 mmol) and 4-(4-(trifluoromethyl)phenoxy)aniline (CAS: 57478-19-0, 662 g, 2.6 mmol) were dissolved in a 3:1 mixture of tetrahydrofuran:isopropanol (20 mL total volume). Concentrated hydrochloric acid (0.1 mL) was added and reaction was heated to 70° C. for 10 h. The reaction was concentrated in vacuo and the residue was dissolved in CH₂Cl₂ and washed with sat. NaHCO₃ sol. (2×) and brine. The organic phase was collected, dried over sodium sulfate (Na₂SO₄), filtered and then concentrated in vacuo. Crude material obtained was purified by recrystallization in boiling CH₃CN or isopropanol to give 515 mg (49%) of 7-fluoro-N-(4-(4-(trifluoromethyl)phenoxy)phenyl)quinazolin-4-amine as clear crystals. ¹H NMR (300 MHz, CDCl₃) δ 8.73 (s, 1H), 7.91 (dd, J=9.2, 5.5 Hz, 1H), 7.77-7.65 (m, 2H), 7.61-7.49 (m, 3H), 7.45 (s, 1H), 7.38-7.21 (m, 1H), 7.16-7.02 (m, 4H). ¹⁹F NMR (282 MHz, CDCl₃) δ −61.72 (s, 3F), −104.15-−104.30 (m, 1F).

Protocol:

Cyt-bd inhibitor screening assay in mycobacteria (M. bovis BCG and M. tuberculosis):

1. Grow parental strain M. bovis BCG or M. tuberculosis in 7H9-ADS-tween 80 medium supplemented with glycerol.

2. Harvest culture at mid-log phase (OD₆₀₀ of 0.2 to 0.5)

3. Wash twice with 7H9-ADS-tween 80 media (without glycerol)

4. Adjust inoculum size to OD₆₀₀ of 0.05

5. In a white 96-well flat bottom plate, dispense 1 μL drug from 100× drug intermediate plate* into each well

6. Dispense 100 μL of culture into each well

7. Place plates in a humidified incubator at 37° C. for 15 hours

8. Remove plates and cool to room temperature

9. Reconstitute BacTiter-Glo™ according to manufacturer's instructions

10. Dispense 25 μL of BacTiter-Glo™ reagent into each well

11. Incubate plate at room temperature in the dark for 12 (between 10 to 20) minutes

12. Take luminescence reading on a microplate reader (Gain: 135; Integration time: 1 sec)

13. Plot a dose response curve and obtain the ATP IC₅₀ values of the test compound in the presence or absence of Q203

Drug Intermediate Plate:

(200× plates):

Using DMSO as a diluent, perform a 2-fold serial dilution of each test compound in a 96-well round bottom plate starting from 5 mM to obtain 10 dilutions

Prepare a 20 μM Q203 stock

(100× plates):

To prepare “compound only” dilution plates, transfer 10 μL of each drug dilution (test compound) from the 200× plate to a fresh plate, and add equal volume of DMSO to each well

To prepare “compound with Q203” dilution plates, transfer 10 μL of each drug dilution (test compound) from the 200× plate to a fresh plate, and add equal volume of 20 μM Q203 stock to each well

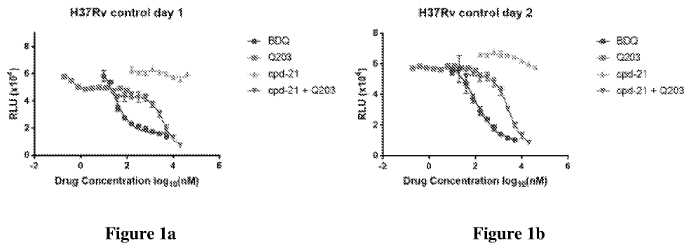

FIGS. 1a-b show data generated with ND-11992 (cpd-21) against H37Rv-Mtb.

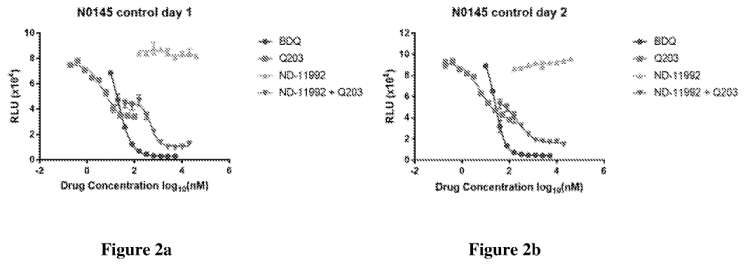

FIGS. 2a-b show data generated with ND-11992 against N0145-Mtb clinical strain.

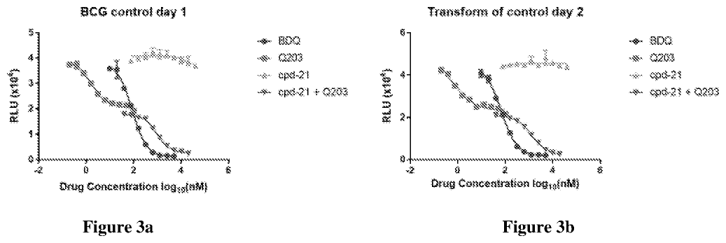

FIGS. 3a-b show data generated with ND-11992 (cpd-21) against M. bovis BCG.

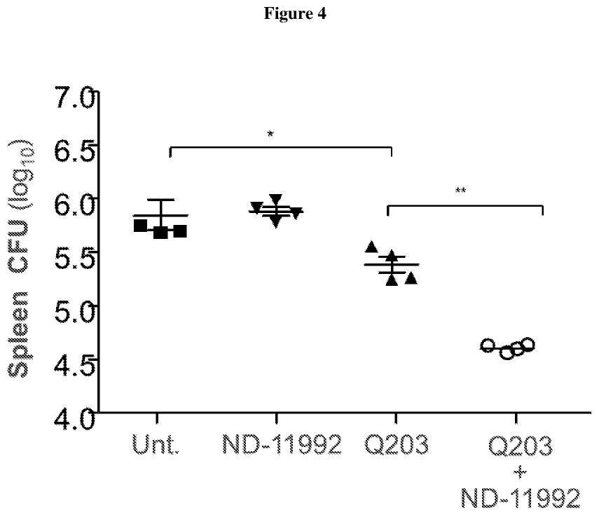

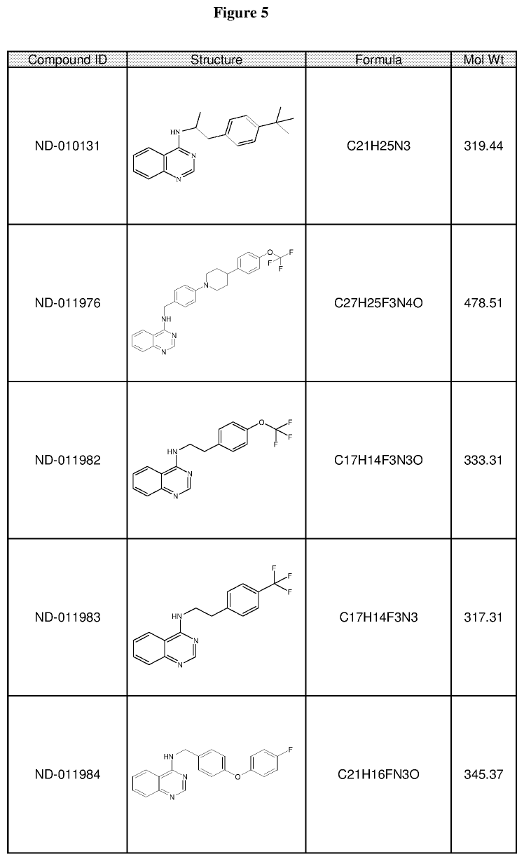

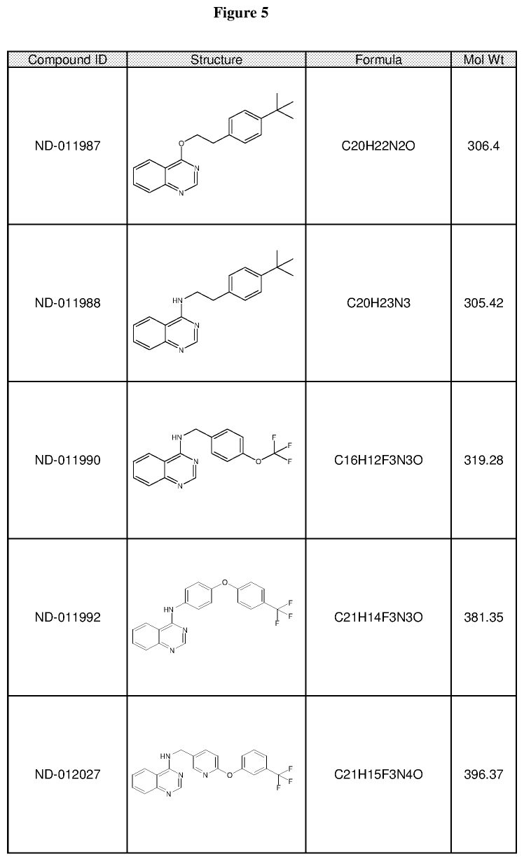

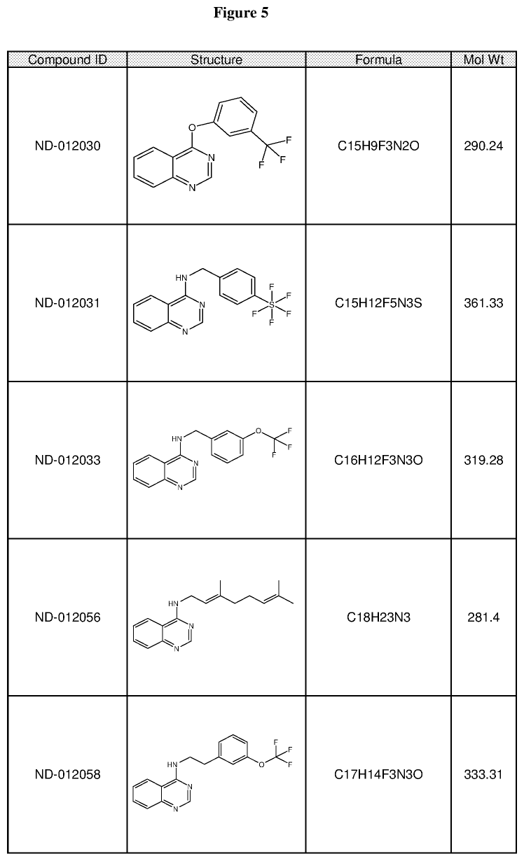

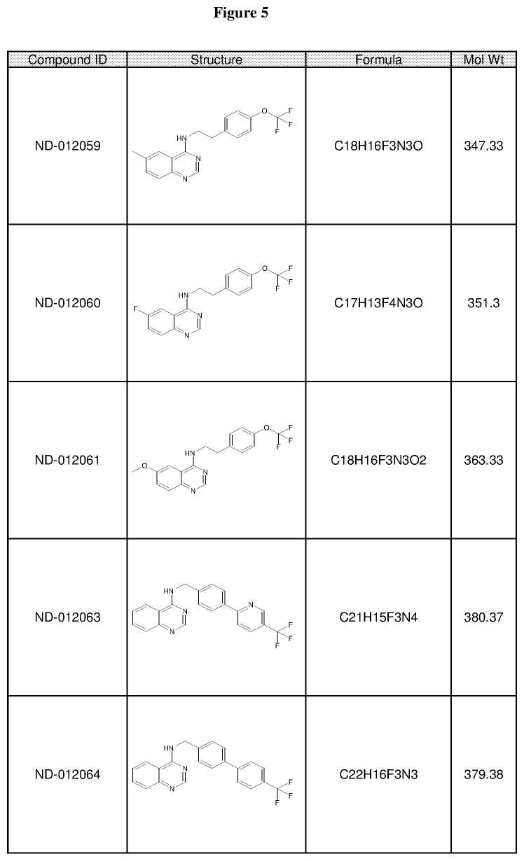

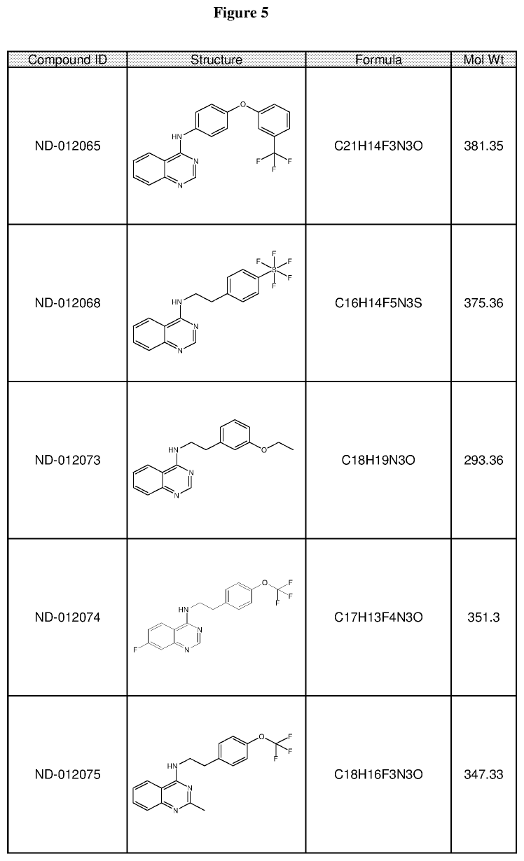

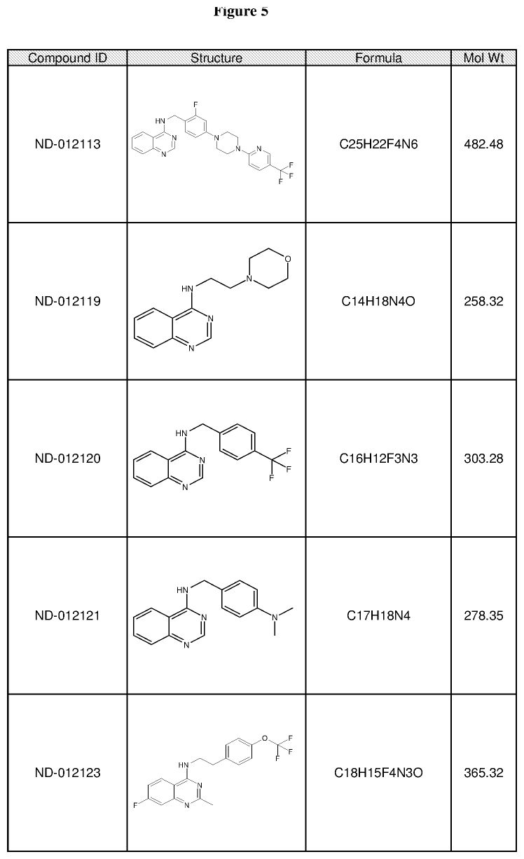

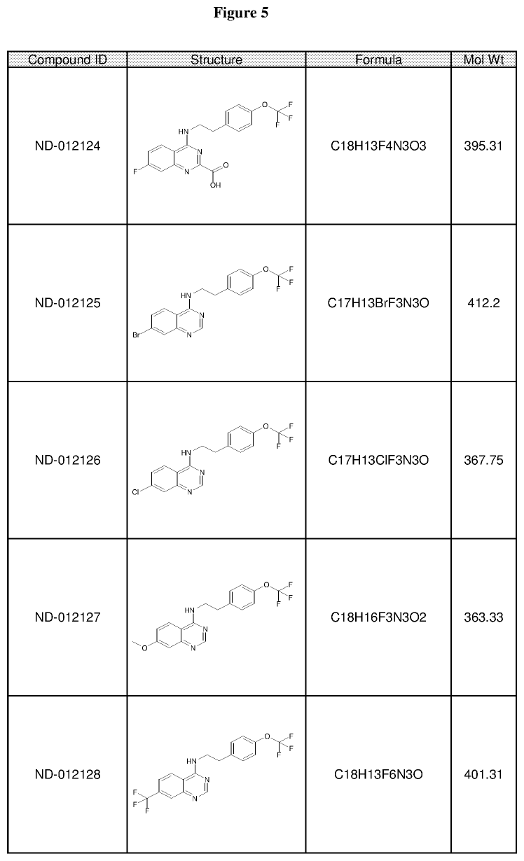

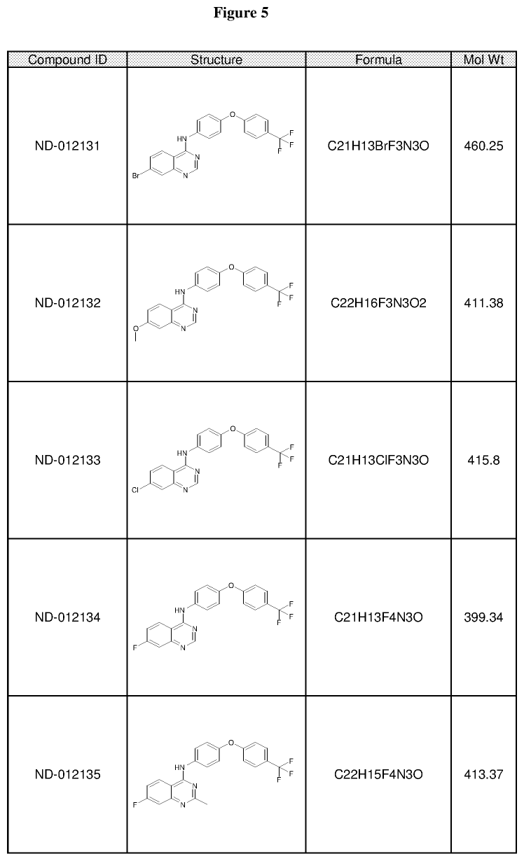

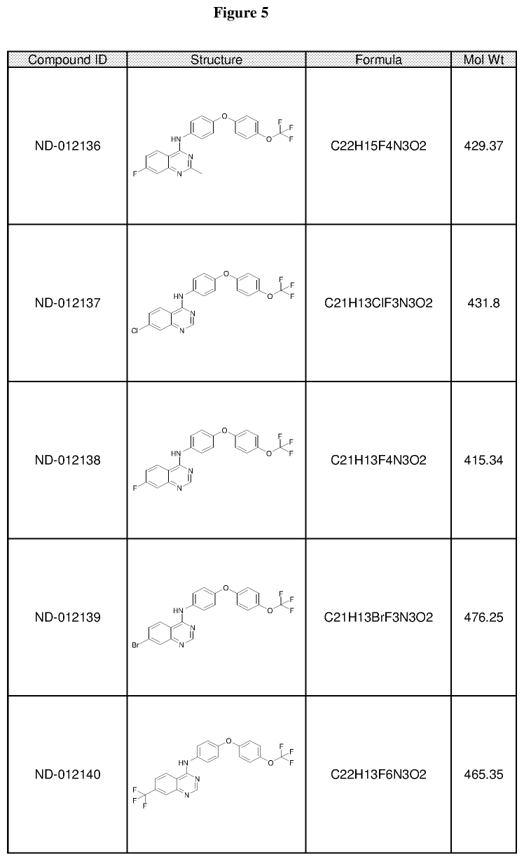

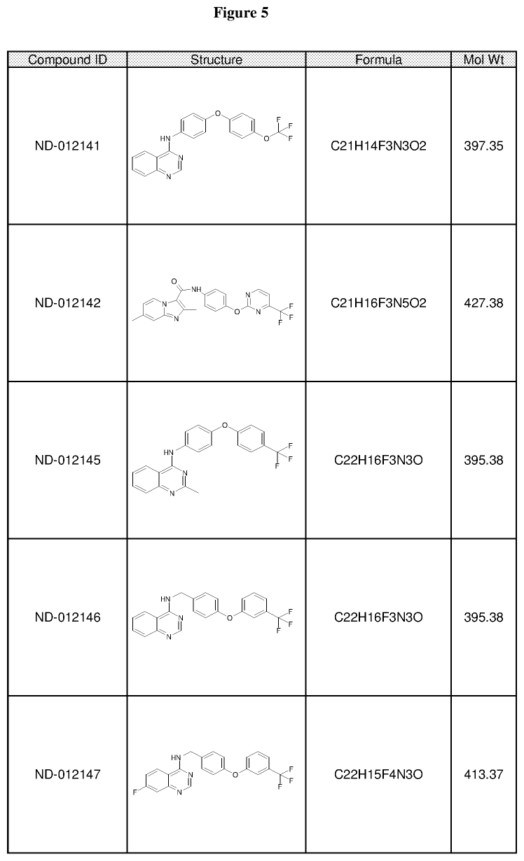

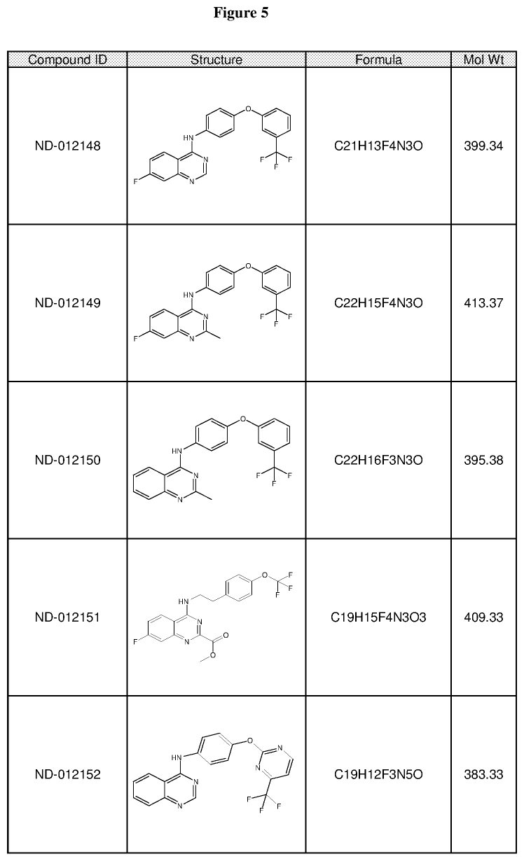

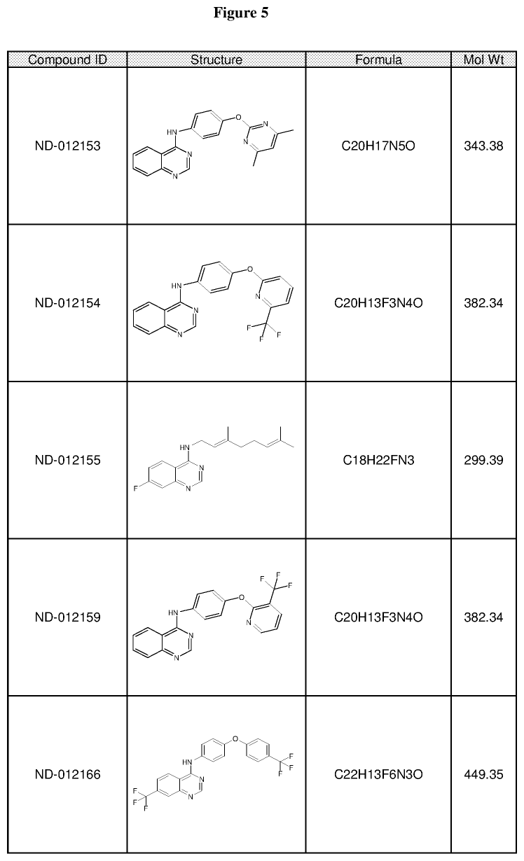

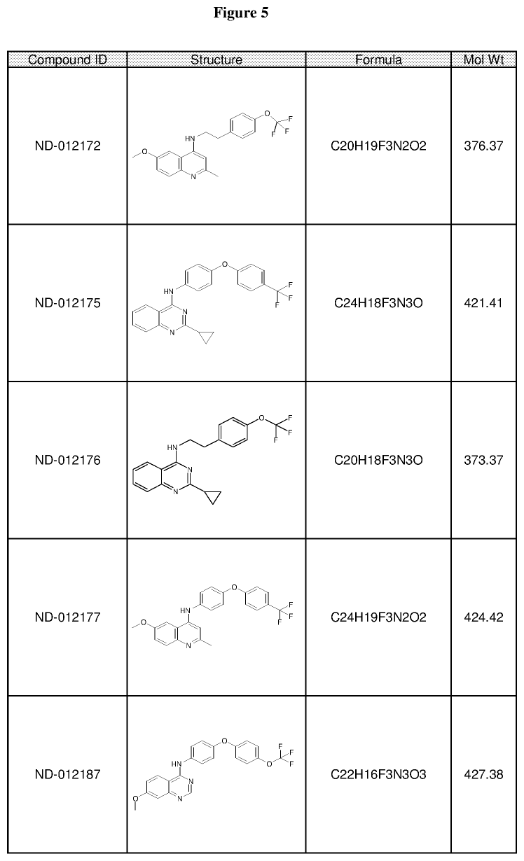

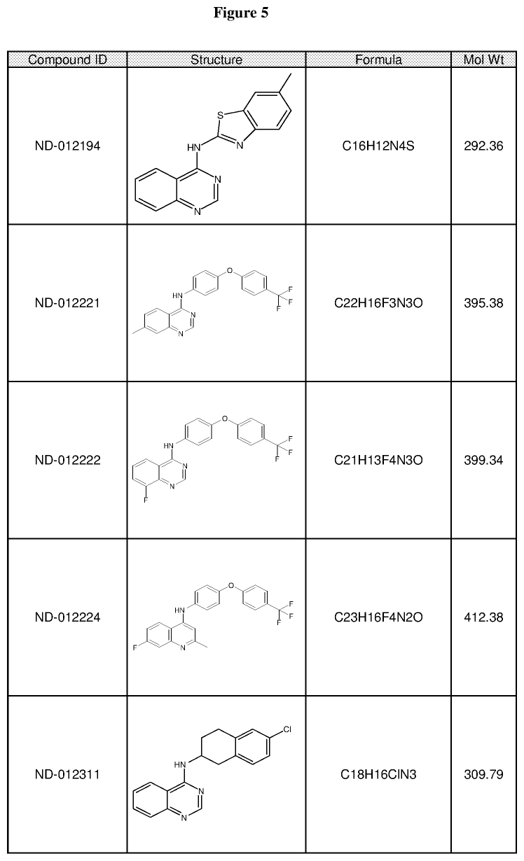

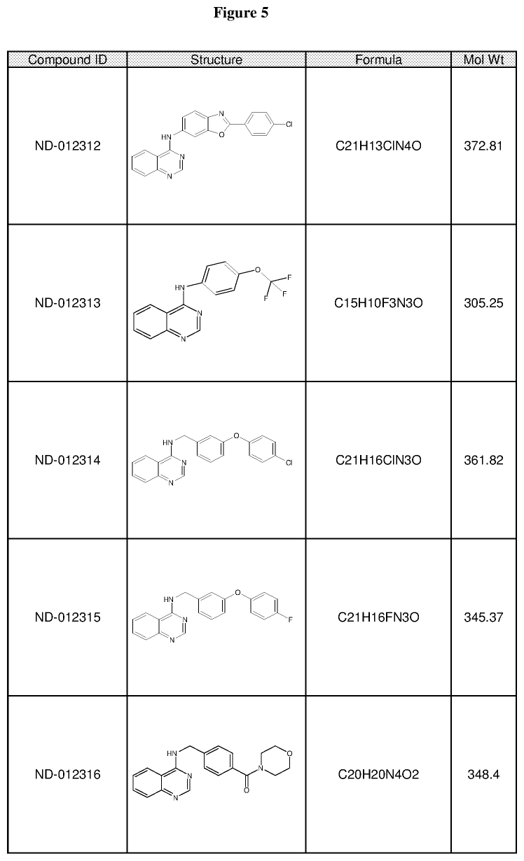

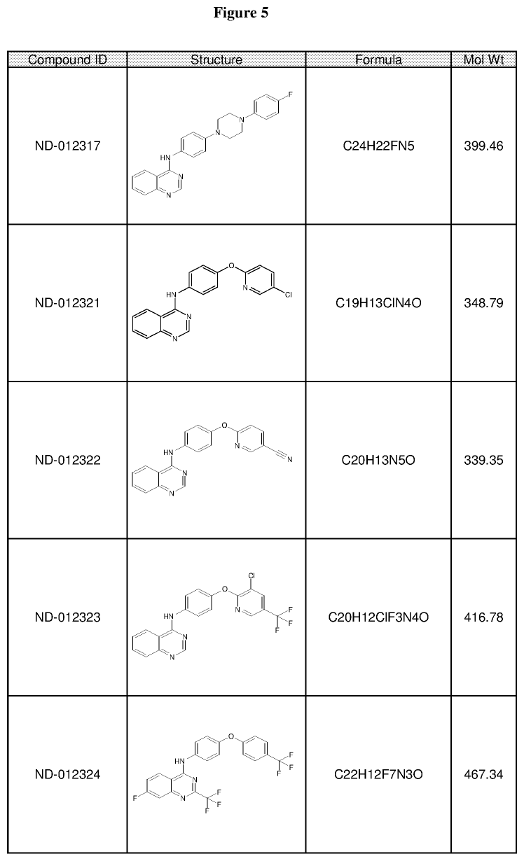

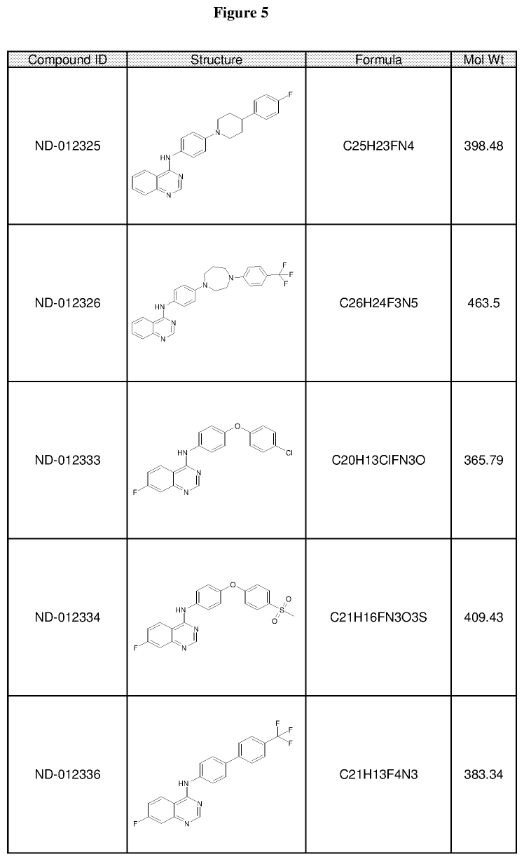

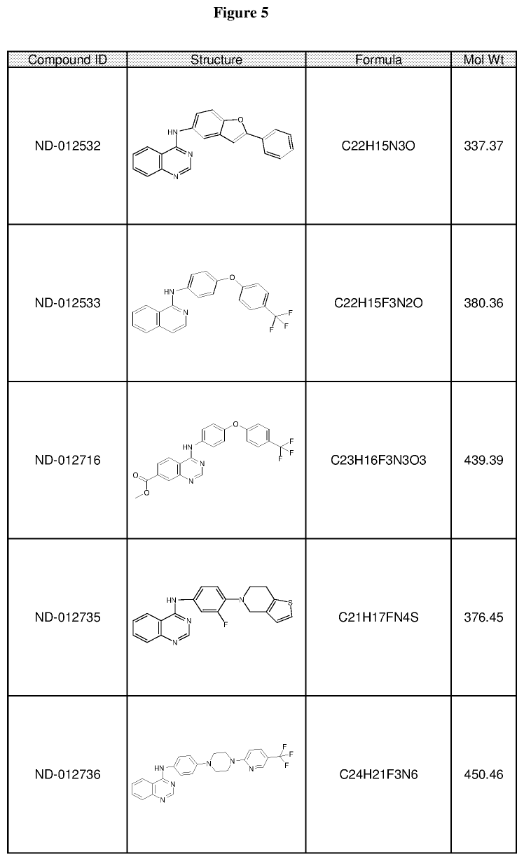

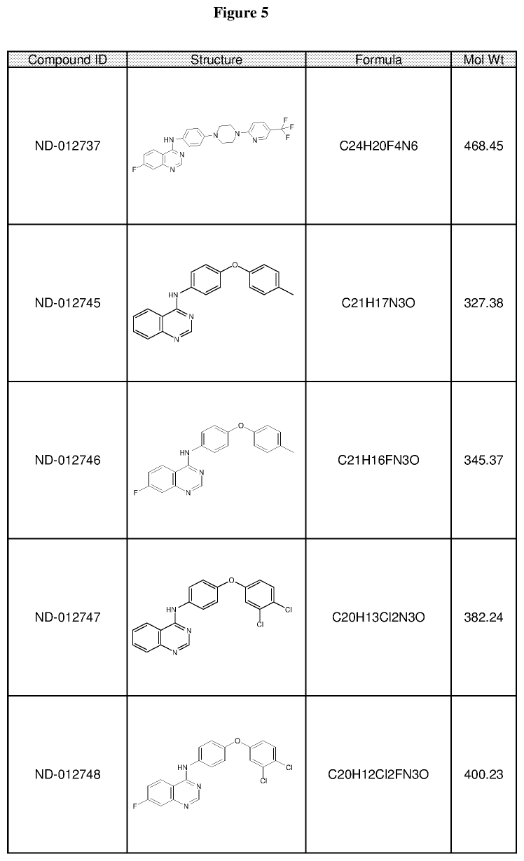

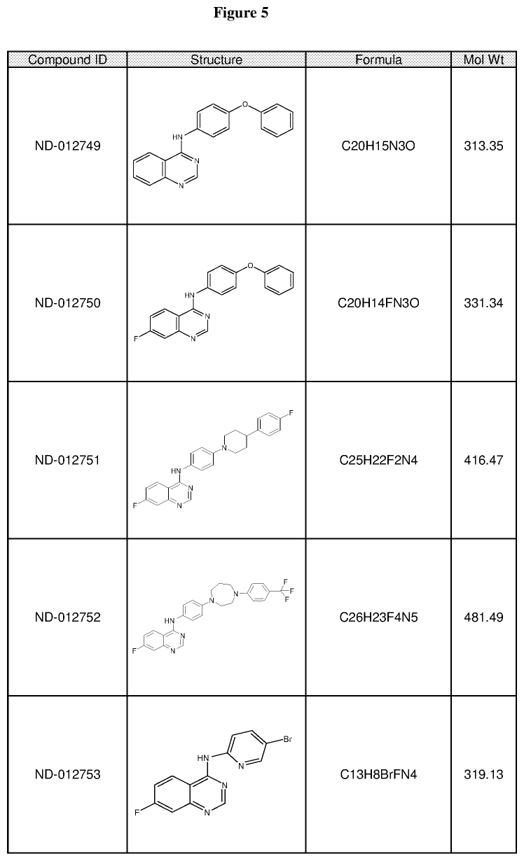

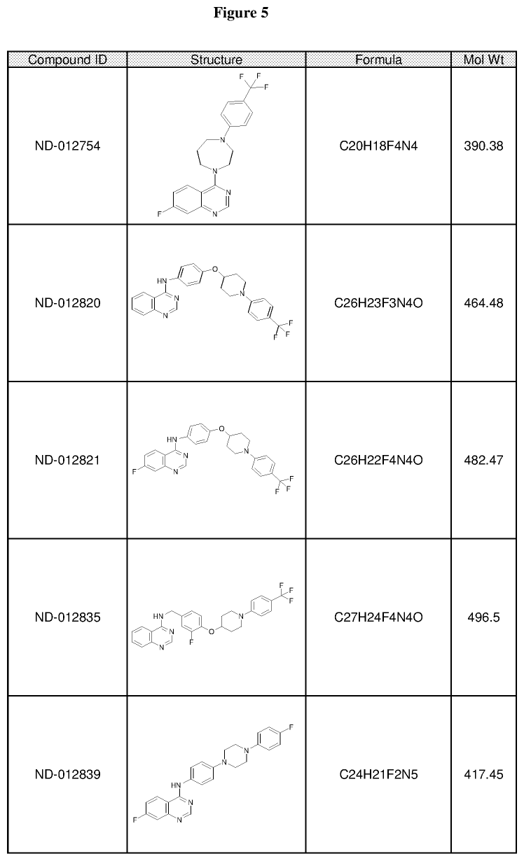

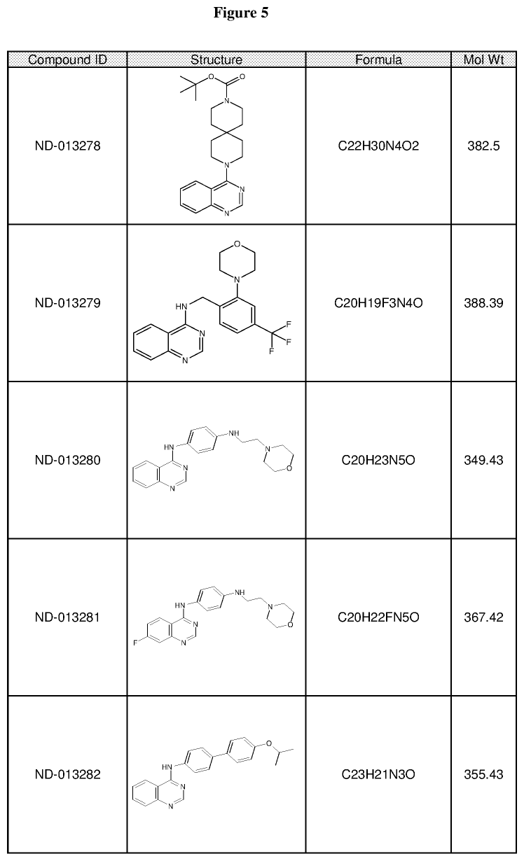

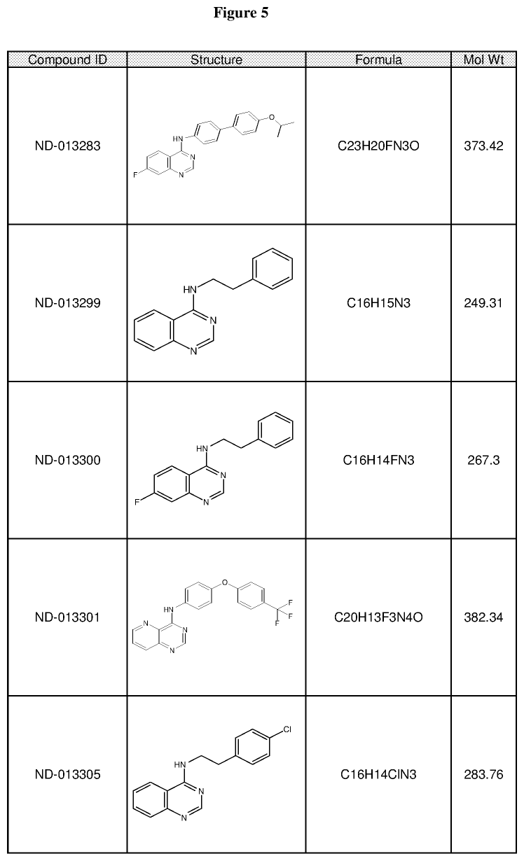

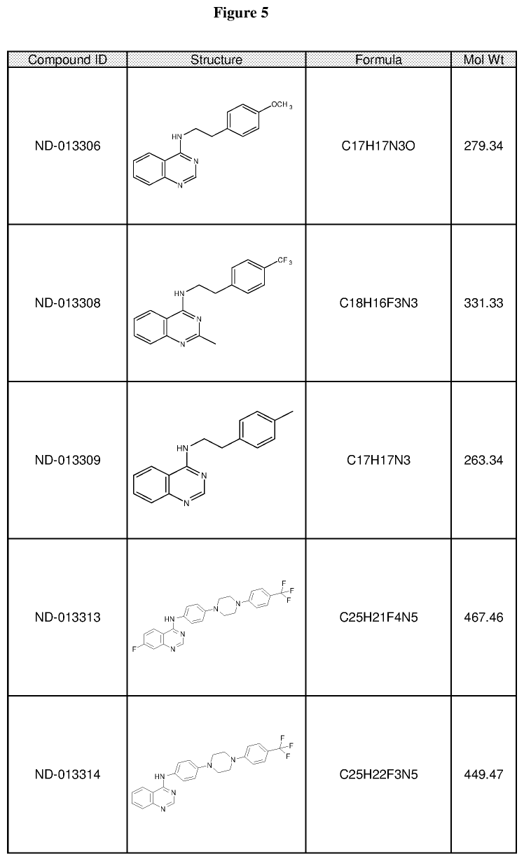

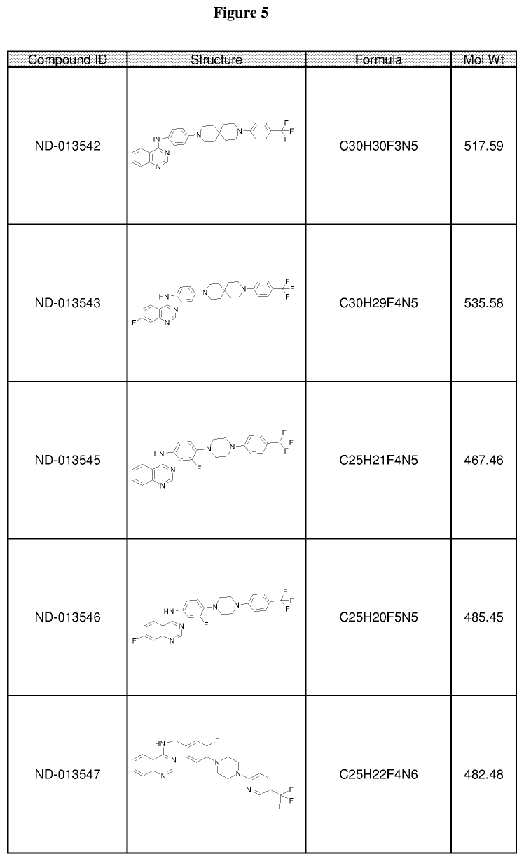

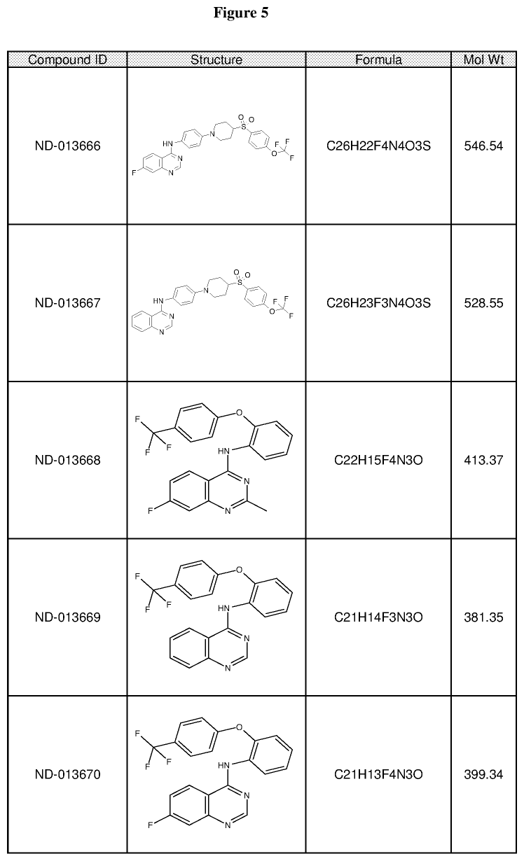

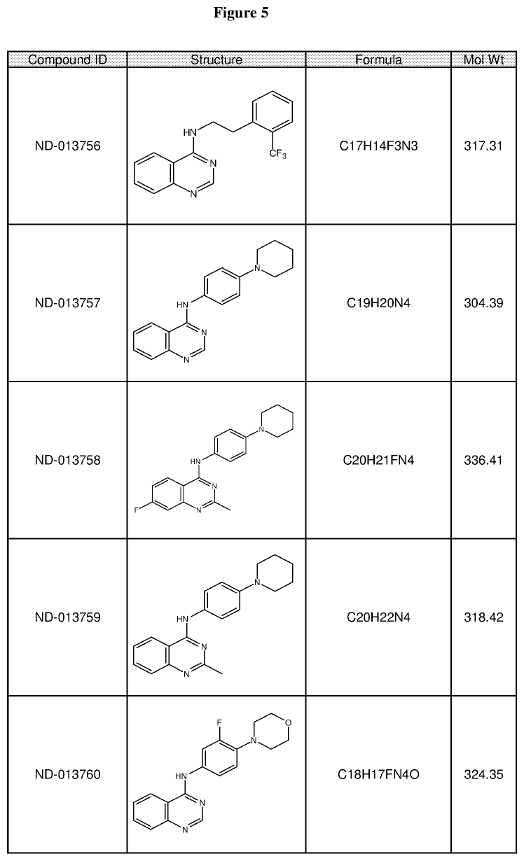

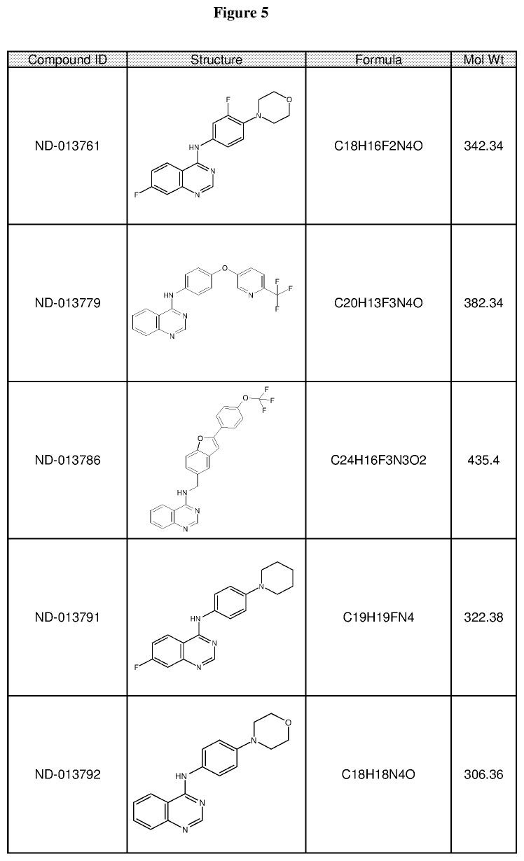

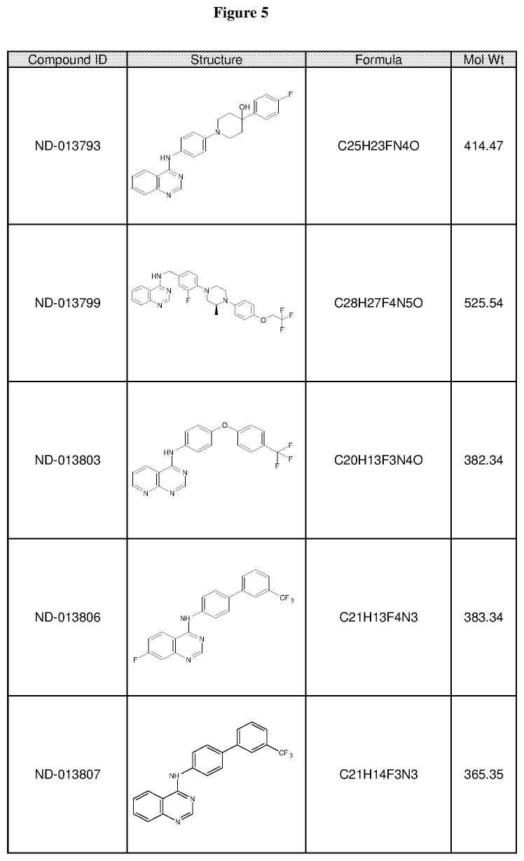

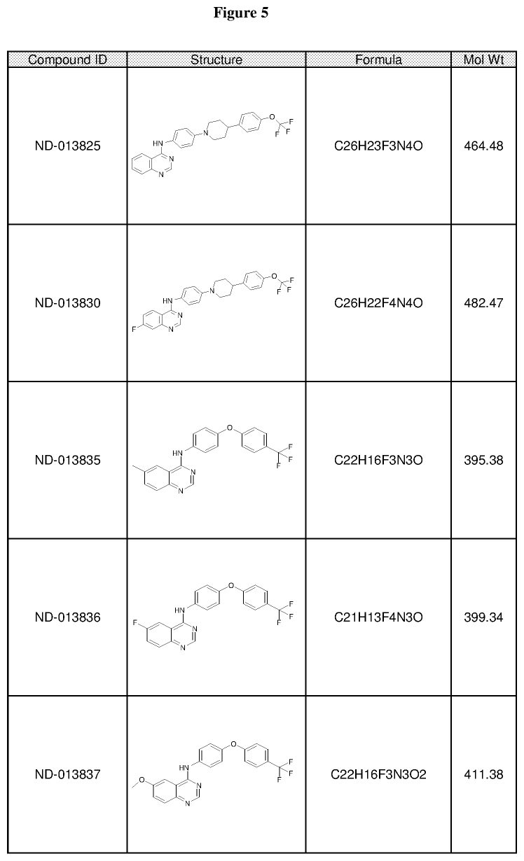

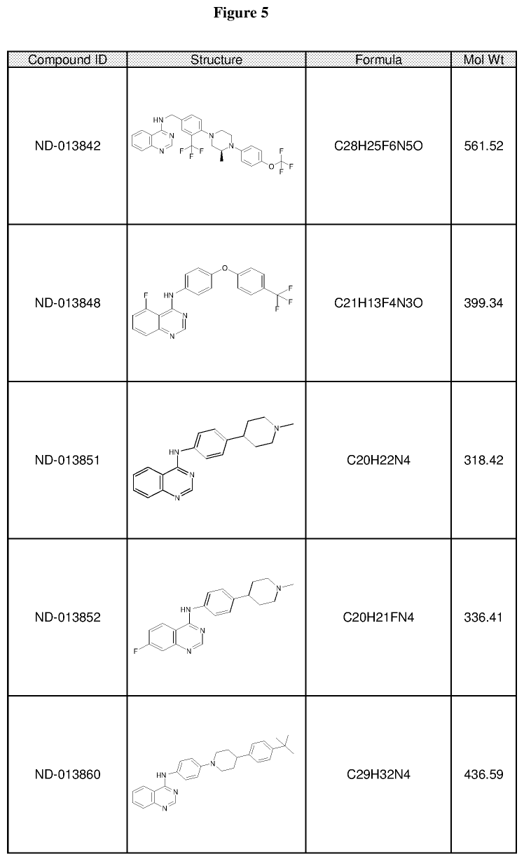

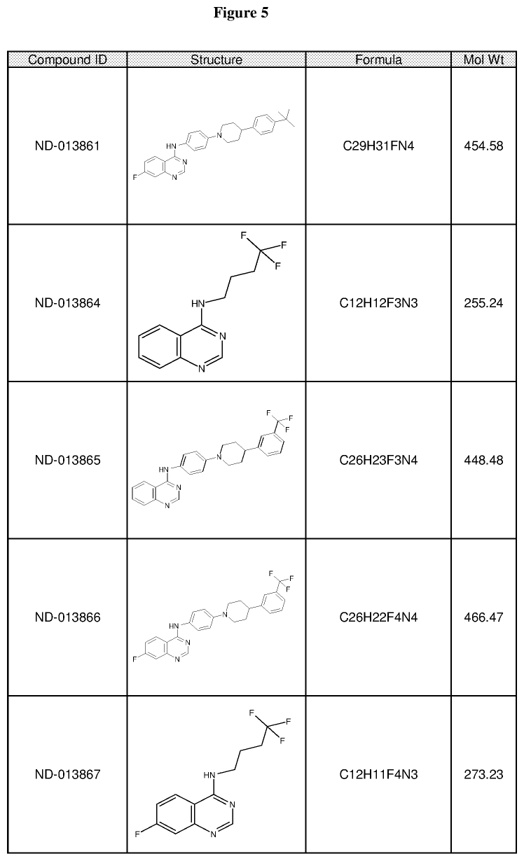

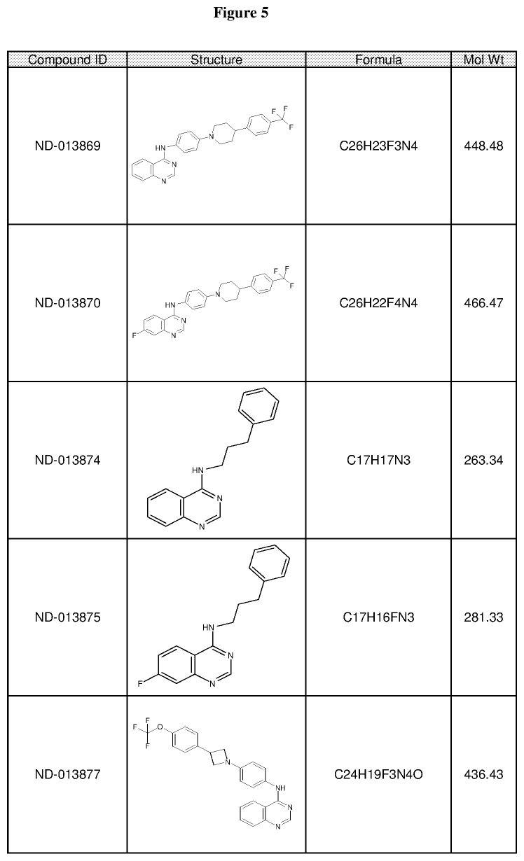

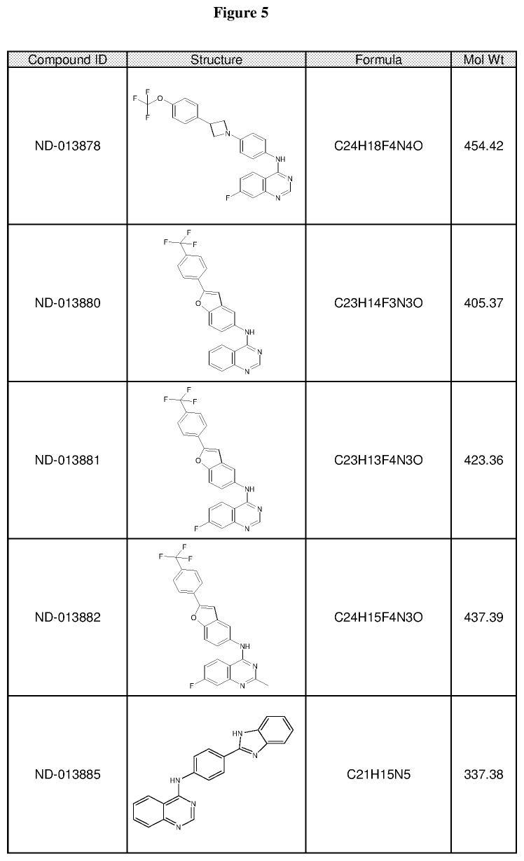

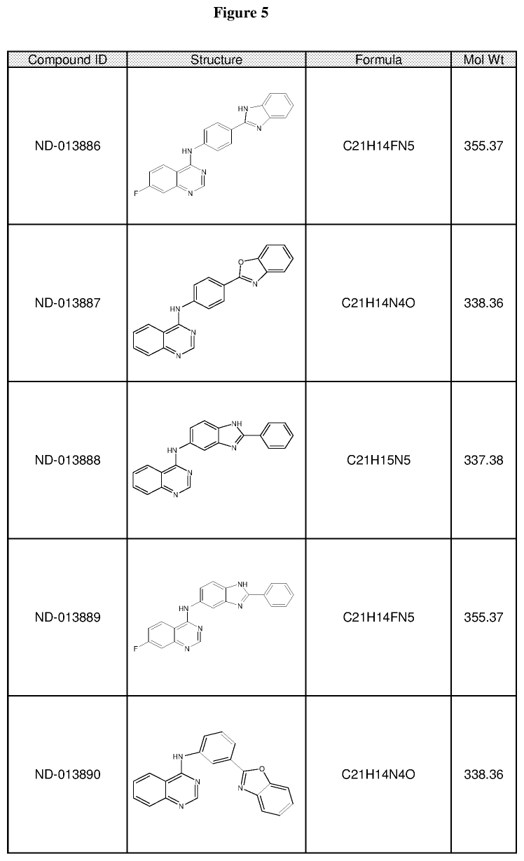

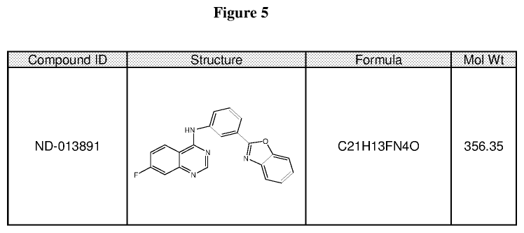

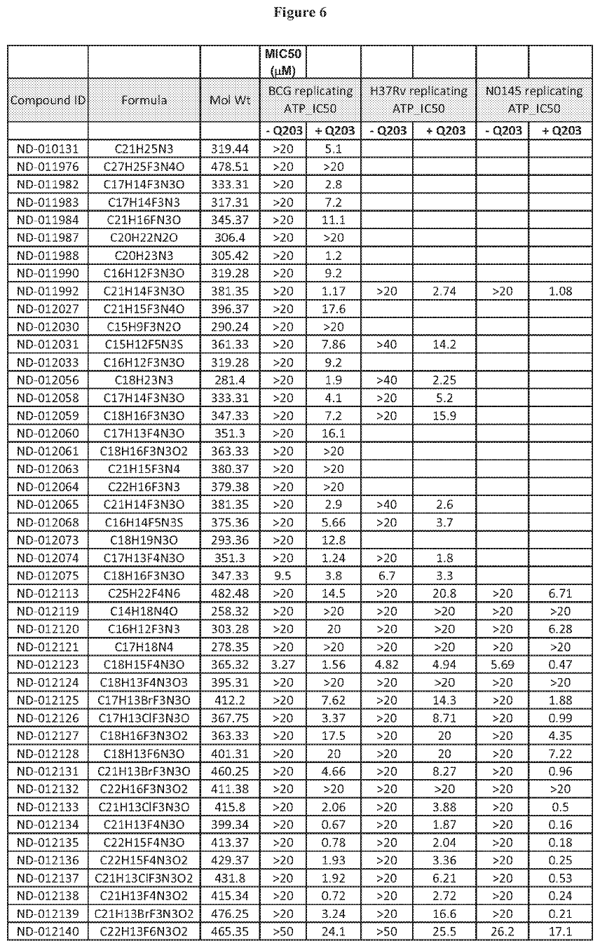

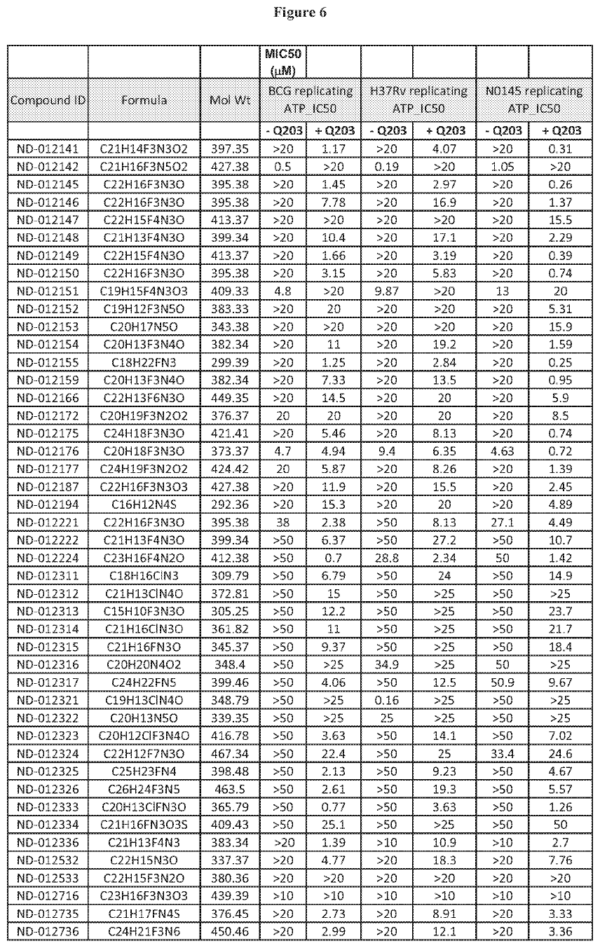

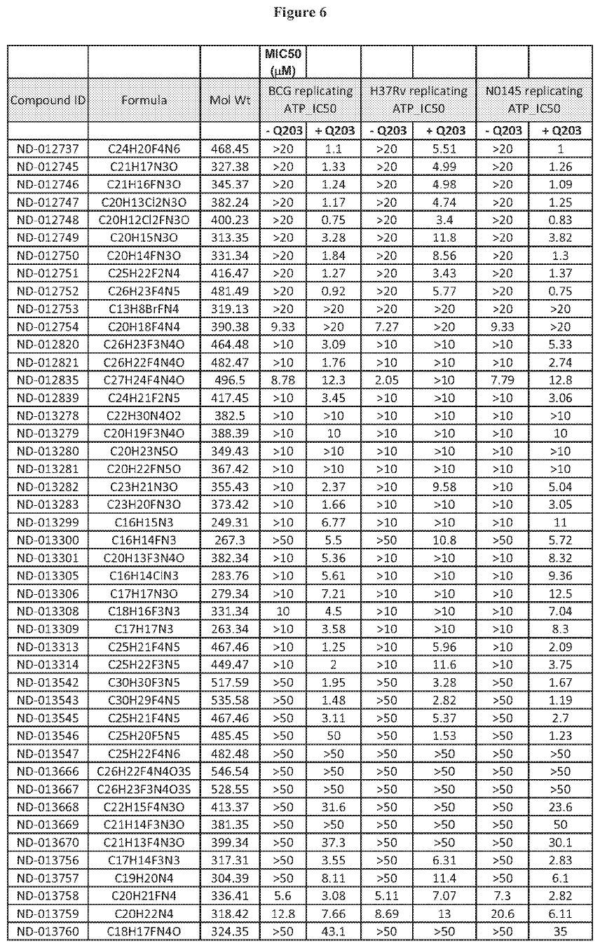

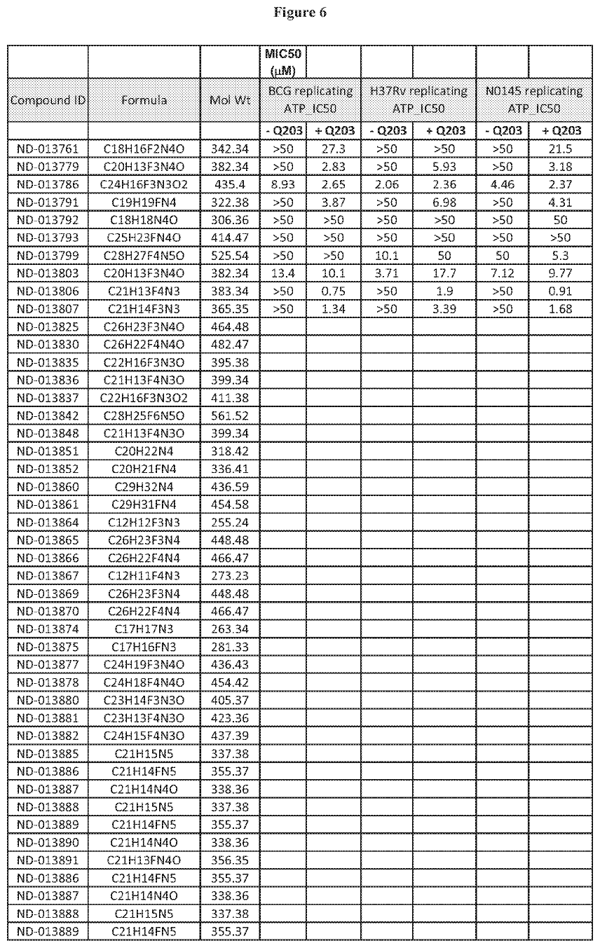

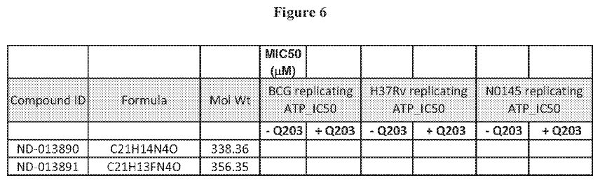

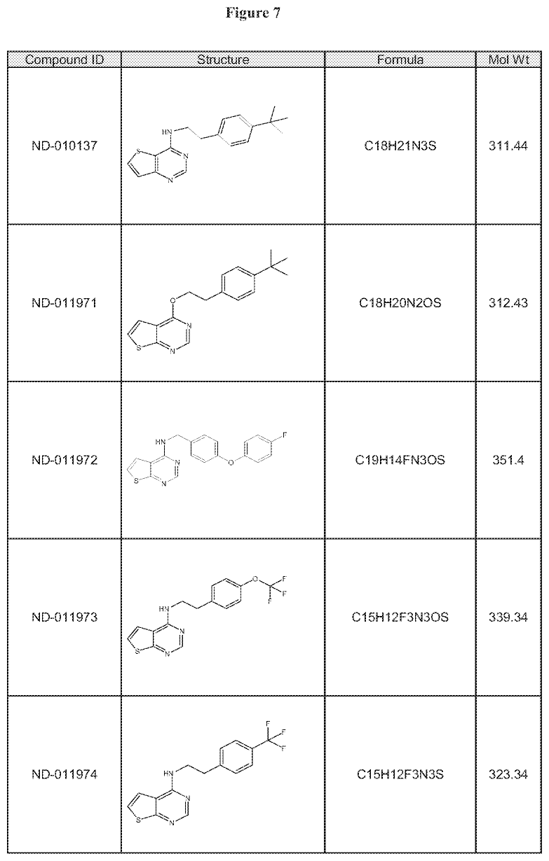

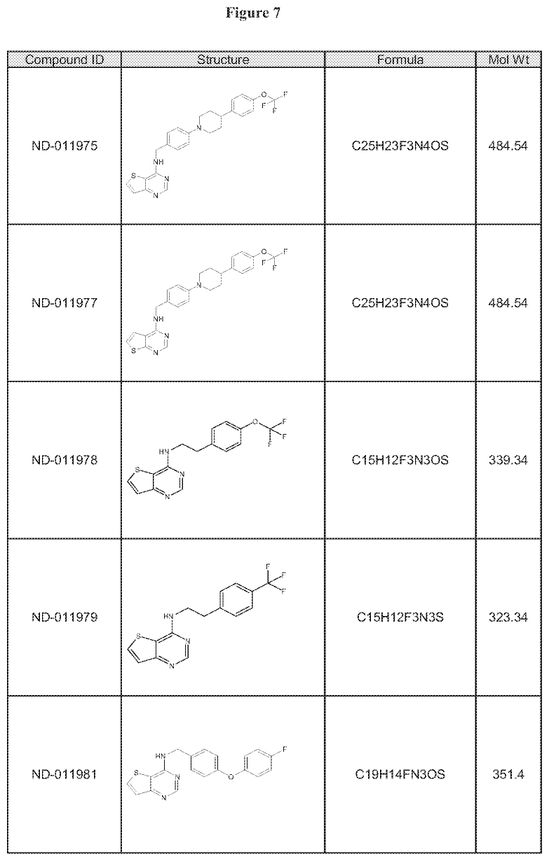

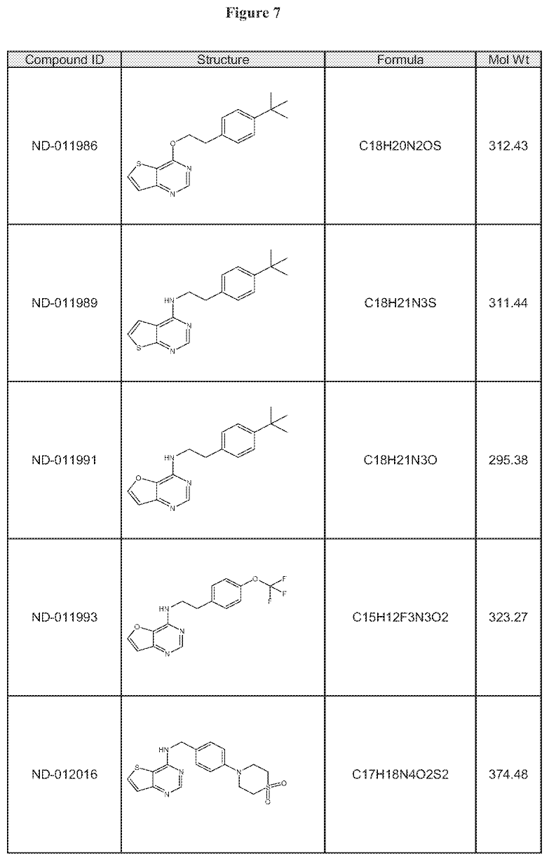

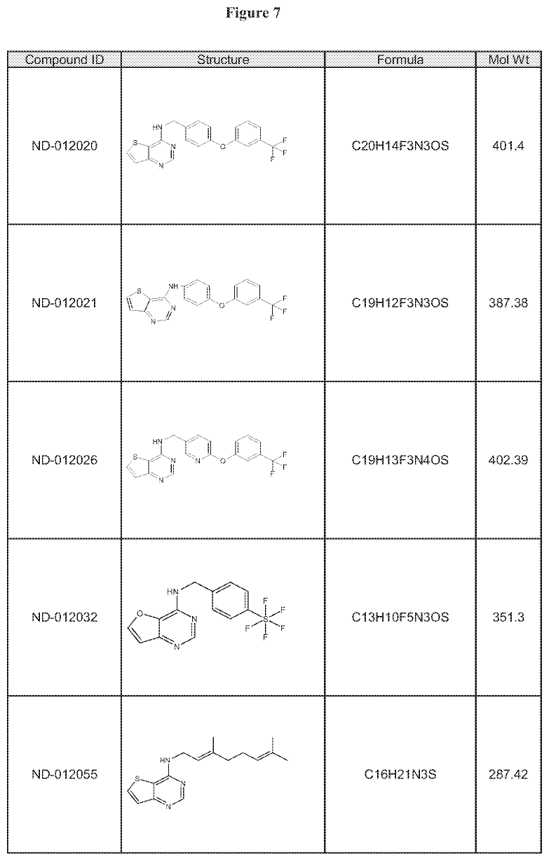

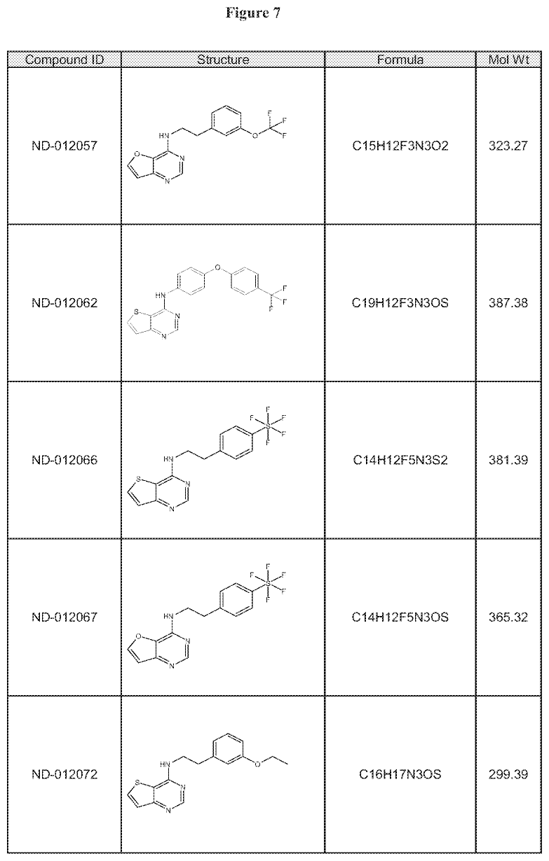

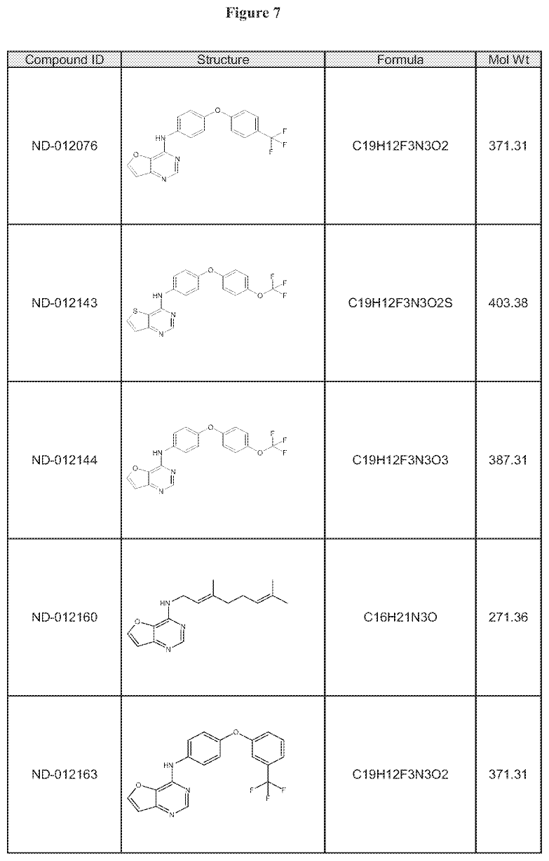

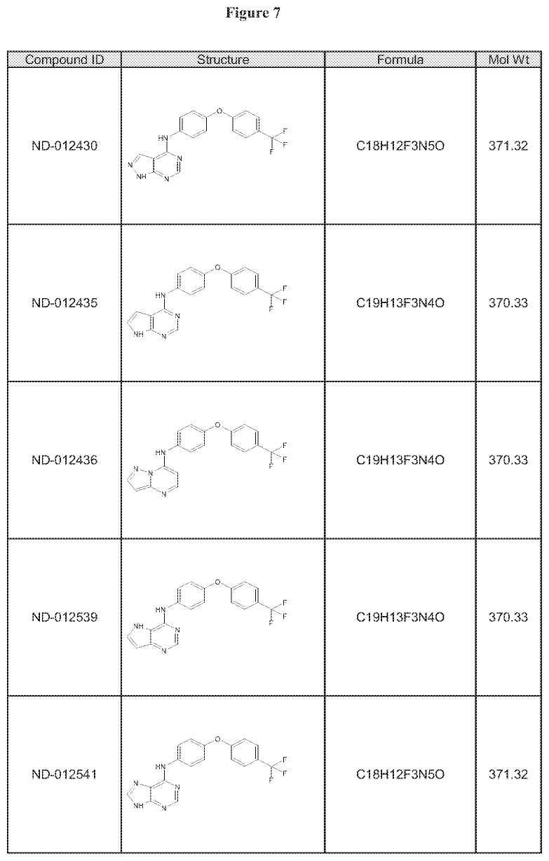

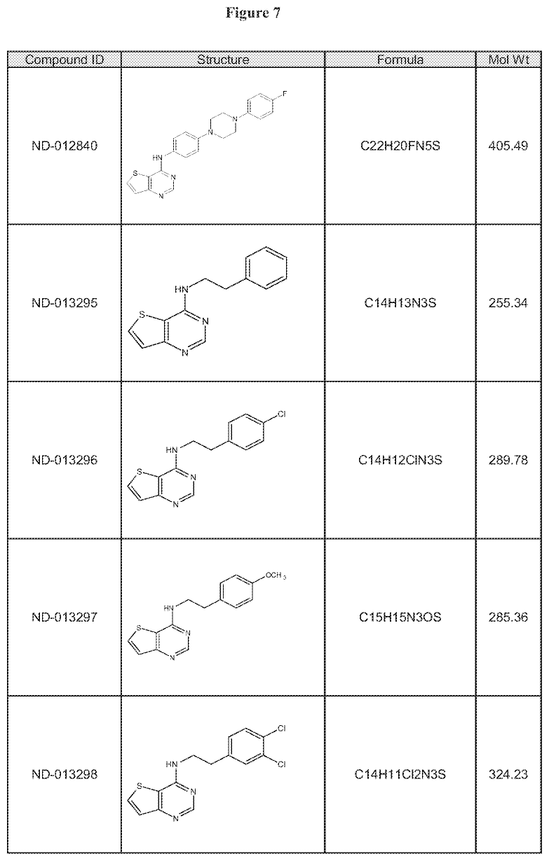

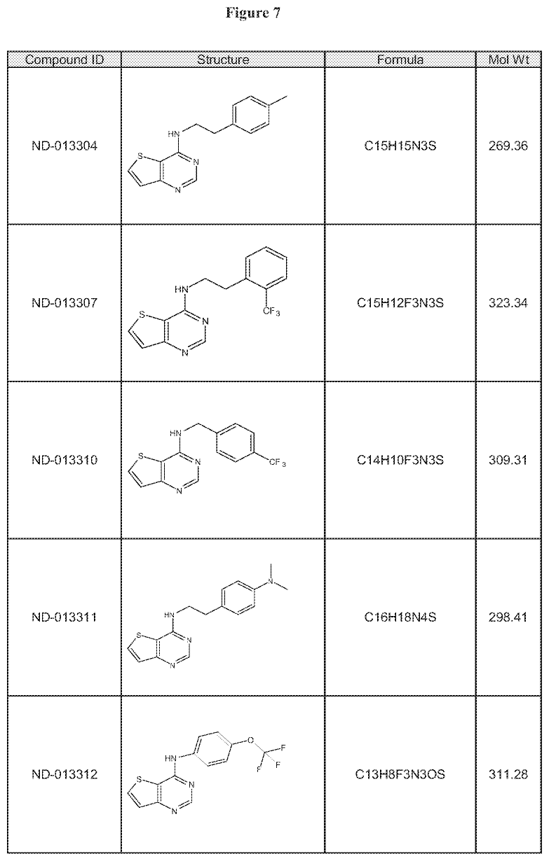

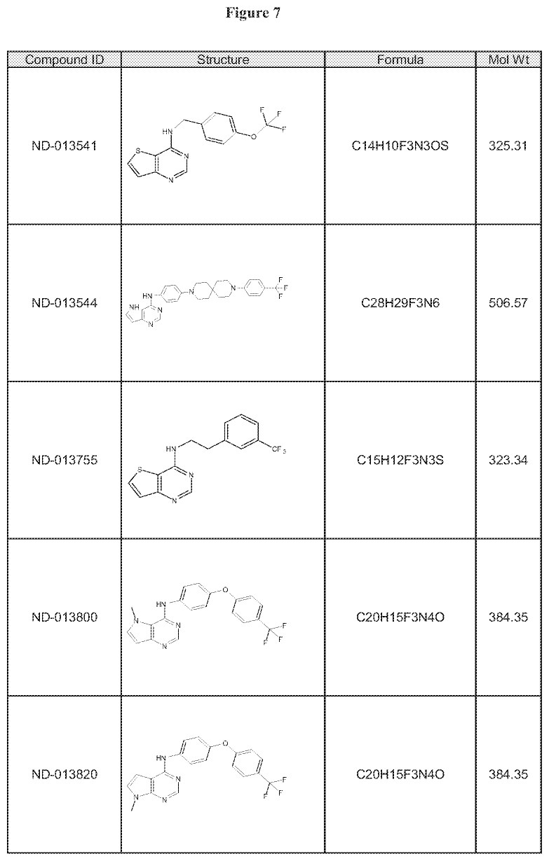

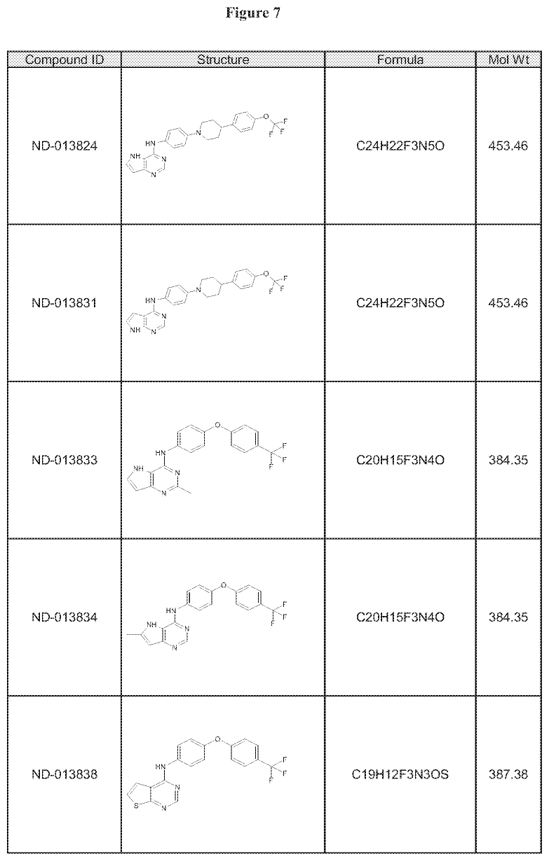

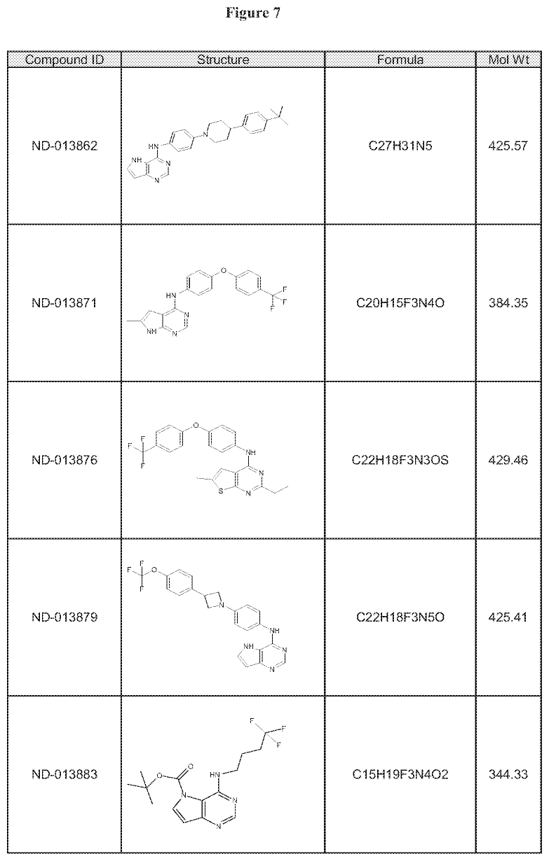

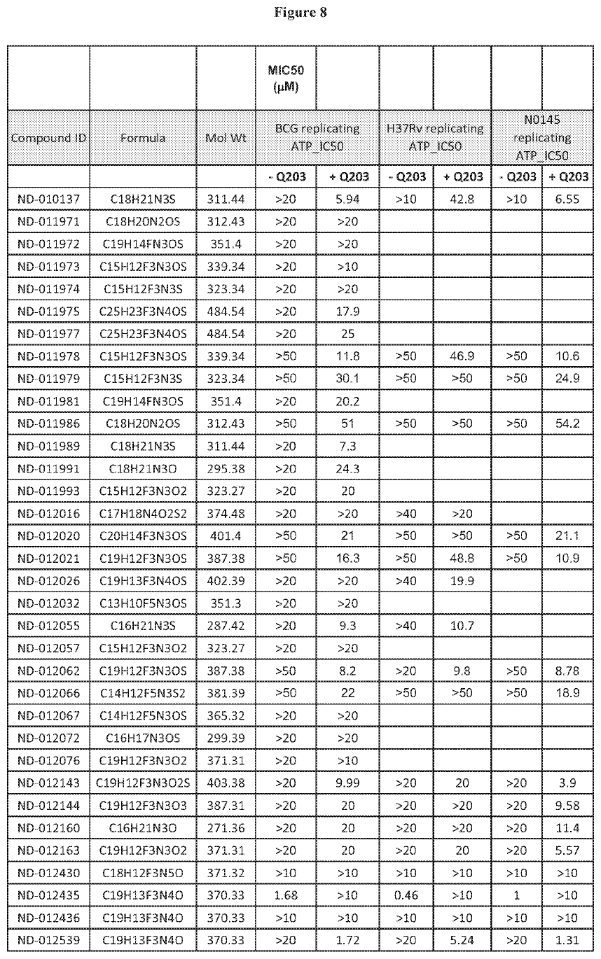

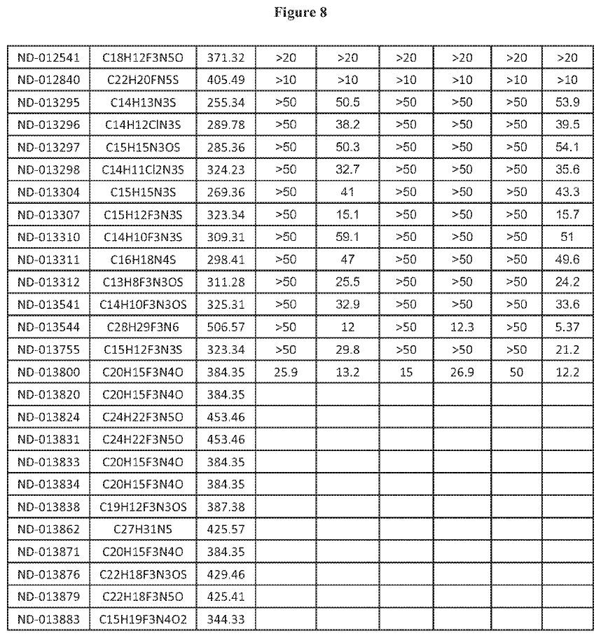

ND-11992 (bd oxidase inhibitor) and Q203 (cyt-bc1:aa3 inhibitor) were evaluated in an acute murine model of M. tuberculosis infection alone and in combination. ND-11992 (10 mg/kg PO)+Q203 (5 mg/kg PO) results in bactericidal efficacy with 1.5 log 10 CFU drop in bacteria when dosed orally for 5 days. Combination therapy was superior to 10 mg/kg of ND-11992 (no CFU drop) or 5 mg/kg of Q203 (0.5 CFU drop). Both compounds were bacteriostatic when dosed alone as single agents. The results are shown in FIG. 4.

ND-11992 (bd oxidase inhibitor) and ND-11598 (cyt-bc1:aa3 inhibitor) were evaluated against various Mycobacterium abscessus clinical specimens in a checkerboard assay. Additive effects on MICs were observed when both drugs were used in combination. MIC values are given in μg/mL.

| | | MIC ND-11598 | ND-11598 MIC | ND-11992 MIC |
|---|---|---|---|---|
| | | alone | (μg/ml) with 5 | (μg/ml) with 5 |
| Specimen | Activity | (μg/ml) | μg/ml ND 11992 | μg/ml of ND-11598 |
| M. abscessus | Additive | >10 | 5 | 5 |
| Clinical | | | | |
| Isolate #8 | | | | |
| M. abscessus | Additive | >10 | 5 | 5 |
| Clinical | | | | |
| Isolate #38 | | | | |
| M. abscessus | Additive | >10 | 10 | 10 |
| Clinical | | | | |
| Isolate #50 | | | | |

ND-11992 (bd oxidase inhibitor) and ND-11598 (cyt-bc1:aa3 inhibitor) were evaluated against various Nontuberculous mycobacteria (NTM) clinical isolates of the Mycobacterium avium-intracellulare complex (MAIC) in a checkerboard assay. Additive and synergistic effects on MICs were observed when both drugs were used in combination. MIC values are given in μg/mL.

| | | MIC ND-11598 | ND-11598 MIC | ND-11992 MIC |
|---|---|---|---|---|
| | | alone | (μg/ml) with 0.5 | (μg/ml) with 0.5 |
| Specimen | Activity | (μg/ml) | μg/ml ND 11992 | μg/ml of ND-11598 |
| NTM Clinical | Additive and | 0.25 | 0.0156 | 0.0625 (ND-11598 |
| Isolate #25 | Synergistic | | | was only 0.125 μg/ml) |
| NTM Clinical | Additive and | 1 | 0.0156 | 0.125 |
| Isolate #50 | Synergistic | | | |
| NTM MAIC A5 | Additive and | 1 | 0.25 | 0.25 |
| (serotype 4) | Synergistic | | | |
| NTM MAIC | Additive and | >10 | 0.25 | 0.5 |
| Serovar #4 | Synergistic | | | |
| NTM MAIC | Additive and | >10 | 1 | 1 |
| Serovar #5 | Synergistic | | | |
| NTM MAIC | Additive | 0.0156 | 0.0078 (with | Not measured - |
| Serovar #6 | | | ND-11992 at 1) | ND-11598 alone |
| | | | | was below limit |
| | | | | of detection |
| NTM MAIC | Additive and | 10 | 0.25 | 0.5 |
| Serovar #10 | Synergistic | | | |

The entire contents of each reference, patent, publication, patent application, and URL described herein is hereby incorporated by reference, the same as if set forth at length.

1. A compound described herein that inhibits or targets Cyt-bd.

2. A pharmaceutical composition comprising the compound of claim 1 and a pharmaceutically acceptable carrier.

3. A compound having any one of the formulas (A)-(E), (A′), (A″), and (B′) described herein, or pharmaceutically acceptable salt thereof.

4. A pharmaceutical composition comprising the compound of claim 3 and a pharmaceutically acceptable carrier.

5. A kit, comprising:
the compound of claim 1; and
one or more Cyt-bc1:aa3 inhibitor or F₁F₀-ATP synthase inhibitor, or
one or more pharmaceutical composition comprising one or more Cyt-bc1:aa3 inhibitor or F₁F₀-ATP synthase inhibitor and a pharmaceutically acceptable carrier.

6. A method, comprising administering the compound of claim 1, to a subject suffering from mycobacterial disease or infection.

7. A method, comprising co-administering, to a subject suffering from mycobacterial disease or infection:
the compound of claim 1; and
one or more Cyt-bc1:aa3 inhibitor or F₁F₀-ATP synthase inhibitor, or
one or more pharmaceutical composition comprising one or more Cyt-bc1:aa3 inhibitor or F₁F₀-ATP synthase inhibitor and a pharmaceutically acceptable carrier.

8. A method, comprising co-administering, to a subject suffering from mycobacterial disease or infection:
the compound of claim 1; and
one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, or
one or more pharmaceutical composition comprising one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, and a pharmaceutically acceptable carrier.

9. A kit, comprising:
the compound of claim 1; and
one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, or
one or more pharmaceutical composition comprising one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, and a pharmaceutically acceptable carrier.

10. A kit, comprising:
the compound of claim 3; and
one or more Cyt-bc1:aa3 inhibitor or F₁F₀-ATP synthase inhibitor, or
one or more pharmaceutical composition comprising one or more Cyt-bc1:aa3 inhibitor or F₁F₀-ATP synthase inhibitor and a pharmaceutically acceptable carrier.

11. A method, comprising administering the compound of claim 3, to a subject suffering from mycobacterial disease or infection.

12. A method, comprising administering the kit of claim 5, to a subject suffering from mycobacterial disease or infection.

13. A method, comprising co-administering, to a subject suffering from mycobacterial disease or infection:
the compound of claim 3; and
one or more Cyt-bc1:aa3 inhibitor or F₁F₀-ATP synthase inhibitor, or
one or more pharmaceutical composition comprising one or more Cyt-bc1:aa3 inhibitor or F₁F₀-ATP synthase inhibitor and a pharmaceutically acceptable carrier.

14. A method, comprising co-administering, to a subject suffering from mycobacterial disease or infection:
the compound of claim 3; and
one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, or
one or more pharmaceutical composition comprising one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, and a pharmaceutically acceptable carrier.

15. A kit, comprising:
the compound of claim 3; and
one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, or
one or more pharmaceutical composition comprising one or more Cyt-bc1:aa3 inhibitor, F₁F₀-ATP synthase inhibitor, NADH dehydrogenase (NDH-2) inhibitor, NADH dehydrogenase (NDH-2) activator or antibacterial agent, and a pharmaceutically acceptable carrier.
